SEQUENCE LISTING (22 sequences)

SEQ ID NO: 1 - protein, 4 aa, Artificial Sequence
  Synthetic peptide
LLQG

SEQ ID NO: 2 - protein, 4 aa, Artificial Sequence
  Synthetic peptide
LAQG

SEQ ID NO: 3 - protein, 4 aa, Artificial Sequence
  Synthetic peptide
LGQG

SEQ ID NO: 4 - protein, 6 aa, Artificial Sequence
  Synthetic peptide
PLAQSH

SEQ ID NO: 5 - protein, 8 aa, Artificial Sequence
  Synthetic peptide
FERQHMDS

SEQ ID NO: 6 - protein, 11 aa, Artificial Sequence
  Synthetic peptide
TEQKLISEEDL

SEQ ID NO: 7 - protein, 7 aa, Artificial Sequence
  Synthetic peptide
GLGQGGG

SEQ ID NO: 8 - protein, 7 aa, Artificial Sequence
  Synthetic peptide
GFGQGGG

SEQ ID NO: 9 - protein, 7 aa, Artificial Sequence
  Synthetic peptide
GVGQGGG

SEQ ID NO: 10 - protein, 7 aa, Artificial Sequence
  Synthetic peptide
GGLQGGG

SEQ ID NO: 11 - protein, 7 aa, Artificial Sequence
  Synthetic peptide
EAQQIVM

SEQ ID NO: 12 - protein, 7 aa, Artificial Sequence
  Synthetic peptide
GGGQLGG

SEQ ID NO: 13 - protein, 7 aa, Artificial Sequence
  Synthetic peptide
GGGQVGG

SEQ ID NO: 14 - protein, 7 aa, Artificial Sequence
  Synthetic peptide
GGGQRGG

SEQ ID NO: 15 - protein, 6 aa, Artificial Sequence
  Synthetic peptide
GQQQLG

SEQ ID NO: 16 - protein, 7 aa, Artificial Sequence
  Synthetic peptide
PNPQLPF

SEQ ID NO: 17 - protein, 7 aa, Artificial Sequence
  Synthetic peptide
PKPQQFM

SEQ ID NO: 18 - protein, 6 aa, Artificial Sequence
  Synthetic peptide
MKHKGS

SEQ ID NO: 19 - protein, 21 aa, Artificial Sequence
  Synthetic peptide
GSGMKETAAARFERAHMDSGS

SEQ ID NO: 20 - protein, 13 aa, Artificial Sequence
  Synthetic peptide
MGGSTKHKIPGGS

SEQ ID NO: 21 - protein, 5 aa, Artificial Sequence
  Synthetic peptide
GGGGG

SEQ ID NO: 22 - protein, 14 aa, Artificial Sequence
  Synthetic peptide
MKHKGSGGGSGGGS

(19) United States
(12) Patent Application Publication
(10) Pub. No.: US 2011/0184147 A1
(43) Pub. Date: Jul. 28, 2011

(54) ENZYME SUBSTRATE FOR LABELING OF PROTEIN

(75) Inventors: Noriho Kamiya, Fukuoka-shi (JP); Masahiro Goto, Fukuoka-shi (JP); Hiroki Abe, Fukuoka-shi (JP)

(73) Assignee: KYUSHU UNIVERSITY, NATIONAL UNIVERSITY CORPORATION, Fukuoka-shi, Fukuoka (JP)

(21) Appl. No.: 13/001,929

(22) PCT Filed: Jul. 3, 2009
(86) PCT No.: PCT/JP2009/062569

§ 371 Date: Apr. 1, 2011

(30) Foreign Application Priority Data
Jul. 4, 2008 (JP) 2008-176104

(51) Int. Cl. C07K 5/08; C07K 5/06; C07K 19/00; C12P 21/02

(52) U.S. Cl. 530/331; 549/223; 546/37; 562/427; 530/408; 530/391.1; 435/68.1

(57) ABSTRACT

The present invention provides a substrate of TGase represented by (fluorescent group)-(linker)-(a portion containing a Gln residue capable of recognition by transglutaminase (TGase))-R, wherein the fluorescent group is fluorescein isothiocyanate (FITC), Texas Red (TE) or dansyl (Dns) or a group derived therefrom; the linker is a group represented by —NH—(CH₂)ₙ—CO— (n is an integer of 1 to 6); the portion containing a Gln residue capable of recognition by TGase is a group derived from a peptide selected from among QG and the like; and R is a hydroxyl group, or biotin, nucleic acid, azide, alkyne, maleimide or cyclopentadiene, or a group derived therefrom.

### TECHNICAL FIELD

The present invention relates to a method of using transglutaminase as a catalyst for conjugating functional molecules to proteins, more particularly, to a method for conjugating flurochromes to antibodies. The present invention also relates to a novel fluorescent substrate of transglutaminase. The present invention can be used for fluorescent labeling of recombinant proteins or antibodies, as well as for preparing antibody arrays through site-specific immobilization.

### BACKGROUND ART

Bioassays for detecting and quantifying a specific substance (e.g. hormone, substrate, protein, or peptide) in a biological sample are analytical methods that have recently become indispensable in not only clinical but also various other fields including histology, molecular biology, and immunology. In particular, immunoassay that depends on an antigen-antibody reaction for recognizing a molecule of interest is a highly sensitive and specific method that assumes a key position among bioassays. Antibodies are actively used in the medical field and their use is studied energetically not only as a targeting device in bioimaging and drug delivery systems (DDS) but also as pharmaceuticals (antibody drugs).

In terms of interaction with other substances (i.e., specificity), antibodies even have better characteristics than enzymes and their repertoires with antigens are so great that they might be called almost infinite. However, being proteins by nature, antibodies are subject to constraint. One problem is that an attempt to introduce a functional molecule into an antibody could potentially result in a loss of its antigen recognizing ability if the functional molecule were introduced into the active center (antigen recognizing site).

A need, therefore, has arisen to develop a technique by which a specific site of an antibody can be selectively modified with a marker without causing a loss of its antigen recognizing ability. A current, common method of modifying antibodies is by chemical modification. However, chemical modification might potentially deactivate the antigen recognizing ability. Another problem is the difficulty in controlling the number of functional molecules to be introduced and the sites of their introduction. Because of this background, it is necessary to develop a novel method of modifying specific sites of antibodies with markers.

Transglutaminase (TGase) is a kind of transferases that catalyze an acyl transfer reaction for binding the γ-carboxyamido group in a particular Gln residue (Q) to various primary amines or a particular Lys residue (K) present in peptides or proteins. If neither primary amines nor Lys residue is present, the water molecule works as an acyl receptor and the Gln residue is deamidated by hydrolysis to become Glu(E). All of these reactions involve dissociation of NH₃, so they are reversible reactions whose equilibrium greatly favors a shift from left to right (Non-Patent Document 1).

TGase is found extensively in nature and among others, GTG derived from guinea pig liver, FTG derived from red sea bream (Pagrus major) and Factor XIII as a human blood clotting factor had been closely studied, and eventually in 1989, microbial TGase (MTG) was discovered. Because of the difficulty in mass production, mammalian TGase had involved a cost problem but MTG, being derived from microorganism, could be produced in large volume by mass culture and hence became available at lower cost. In addition, the molecular weight of MTG is 38,000 and smaller than that of the mammalian TGase (ca. 80,000). Further in addition, the mammalian TGase requires Ca²⁺ to express its activity but MTG does not require Ca²⁺ for activity expression. As another problem, the activity for Gln hydrolysis as a side reaction is lower in MTG than in the mammalian TGase. Thus, MTG has advantageous characteristics that favor industrial use and active studies have recently been made on MTG.

The active center of MTG is Cys64 which, when combined with Asp255 and His274 present in the neighborhood, is assumed to cause MTG to express its catalytic activity. In a putative reaction mechanism of MTG, the thiolate ion of Cys64 performs nucleophilic attack on the side chain of the acyl donor Gln residue, not only forming a thioester active intermediate but also releasing ammonia. Subsequently, an acy receptor such as the side chain of the Lys residue comes proximate to the active center and performs nucleophilic attack on the active intermediate, whereby the resulting product is released and the reaction ends (Non-Patent Document 2).

Several studies have been made on the substrate recognition by TGase.

For substrate recognition of the Gln side, Ando et al. varied the amino acids in the neighborhood of Gln in Z-QG known as a good substrate of TGase and performed an evaluation of reactivity. They reported the following results: during the TGase-catalyzed reaction, an activity drop was found to occur in esterification of the C terminal and introduction of Gly and Gln at the C and N terminals of Gln; the deletion of Gly, variation of Gln to Asn, and the deletion of the Z group were not recognized by TGase (Non-Patent Document 3). Otsuka et al. synthesized a polypeptide consisting of 7 residues with alterations in the sequence of amino acids in the neighborhood of Gln and reacted it with monodansylcadaverine, a TGase acyl acceptor fluorescent reagent, to study the substrate specificity in the neighborhood of the Gln residue. As a result, it was shown that TGase prefers a bulky and highly hydrophobic environment on the N terminal side of the Gln residue whereas it prefers an amino acid of small steric hindrance on the C terminal side (Non-Patent Document 4).

Referring now to IgG antibodies, it is known that they are cut near the hinge region upon limited degradation catalyzed by proteases such as papain, pepsin, and trypsin; Pedersen et al. reported that TGase with a similar structure to the Cys protease papain in terms of active center showed a catalytic mechanism similar to the reverse reaction of protease-catalyzed hydrolysis (Non-Patent Document 5). In addition, Makarova et al. speculated that a eukaryotic TGase might have evolved from proteases (Non-Patent Document 6). The fact that TGase performs substrate recognition in a similar manner to proteases has also been supported by the similarity between the site of a protein at which it was subjected to limited degradation by a protease and the site of recognition by TGase (Non-Patent Document 7).

And up until now, there have been reported several studies on the introduction of a functional molecule into proteins by means of TGase which catalyzes the crosslinking reaction.

According to Tominaga et al., an amidated DNA was introduced by a chemical technique at the C terminal of N-carbobenzyloxyglutaminylglycine (Z-QG) known as a good substrate of MTG whereas a Lys tag was introduced into an enzyme by a genetic engineering technique and the two were crosslinked by MTG, whereupon a protein-DNA hybrid was successfully prepared (Patent Document 1) and this success was applied to DNA-directed immobilization (DDI) (Non-Patent Document 8).

Sato et al. successfully performed MTG-catalyzed crosslinking of a Gln residue in a protein and an amine derivative bound to PEG (Non-Patent Document 9).

According to Lin et al., a protein having a Gln tag introduced by a genetic engineering technique was expressed on a cell surface and a primary amine fluorescent probe was successfully introduced into the protein by MTG; they applied the success to the behavior analysis and imaging of proteins within cells (Non-Patent Document 10).

Further, Mindt et al. made a study on the use of TGase to modify IgG via glutamine or lysine (Non-Patent Document 11).

Patent Document 1: JP 2008-54658A

Non-Patent Document 1: M. Griffin, R. Casadio, C. M. Bergamini, Biochem. J., 368, 377-396 (2002)

Non-Patent Document 2: T. Kashiwagi, K. Yokoyama, K. Ishikawa, K. Ono, D. Ejima, H. Matsui, E. Suzuki, J. Biol. Chem., 277, 44252-44260 (2002)

Non-Patent Document 3: H. Ando, M. Adachi, K. Umeda, A. Matsuura, M. Nonaka, R. Uchio, H. Tanaka, M. Motoki, Agric. Biol. Chem., 53, 2613-2617 (1989)

Non-Patent Document 4: T. Ohtsuka, M. Ota, N. Nio, M. Motoki, Biosci. Biotechnol. Biochem., 64, 2608-2613 (2000)

Non-Patent Document 5: L. C. Pedersen, V. C. Yee, P. D. Bishop, I. Le Trong, D. C. Teller, R. E. Stenkamp, Protein Sci., 3, 1131-1135 (1994)

Non-Patent Document 6: K. S. Makarova, L. Aravind, E. V. Koonin, Protein Sci., 8, 1714-1719 (1999)

Non-Patent Document 7: A. Fontana, B. Spolaore, A. Mero, F. M. Veronese, Adv. Drug Deliv. Rev., 60, 13-28 (2008)

Non-Patent Document 8: J. Tominaga, Y. Kemori, Y. Tanaka, T. Maruyama, N. Kamiya, M. Goto, Chem. Commun., 401-403 (2007)

Non-Patent Document 9: H. Sato, E. Hayashi, N. Yamada, M. Yatagai, Y. Takahara, Bioconjugate. Chem., 12, 701-710 (2001)

Non-Patent Document 10: C. Lin, A. Y. Ting., J. Am. Chem. Soc., 128, 4542-4543 (2006)

Non-Patent Document 11: Thomas L. Mindt et. al., Bioconjugate Chem., 19, 271-278 (2008)

### SUMMARY OF THE INVENTION

The present inventors made an attempt for application of MTG to the modification of antibodies. In their study, fluorescence featuring high stability and high resolution was selected as a marker and a fluorescent group bound to Gln and Gly via a linker was used as a fluorescent substrate of MTG. As a result, a novel modification method allowing for a wide range of applications was developed; in addition to the ability to perform fluorescent labeling of recombinant proteins in a manner selective for the site of tag introduction, the method is capable of direct MTG-catalyzed introduction of the fluorescent substrate into an antibody itself without requiring a genetic engineering recombination step such as tag introduction; this has led to the accomplishment of the present invention.

The present invention provides the following:
1) A substrate of TGase represented by (fluorescent group)-(linker)-(a portion containing a Gln residue capable of recognition by transglutaminase (TGase))-R
   (wherein:

the fluorescent group is fluorescein isothiocyanate (FITC), Texas Red (TE) or dansyl (Dns) or a group derived therefrom;

the linker is a group represented by —NH—(CH₂)ₙ—CO— (n is an integer of 1 to 6);

the portion containing a Gln residue capable of recognition by TGase is QX (X is an amino acid residue other than lysine (Lys)); and

R is a hydroxyl group, or biotin, nucleic acid, polyethylene glycol, azide, alkyne, maleimide or cyclopentadiene, or a group derived therefrom.)
2) The substrate of TGase according to 1) wherein the portion containing a Gln residue capable of recognition by TGase is QG and R is a hydroxyl group or a group derived from biotin or alkyne.
3) The substrate of TGase according to 2) which is one member of the group consisting of:

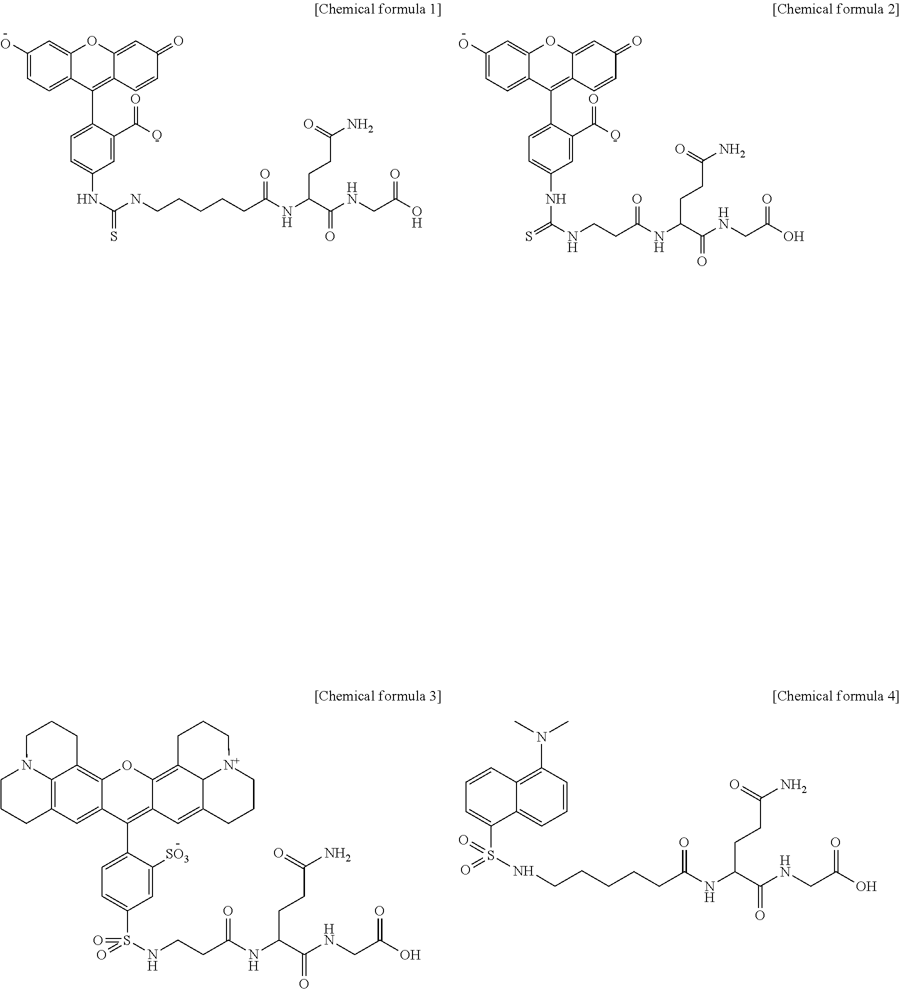

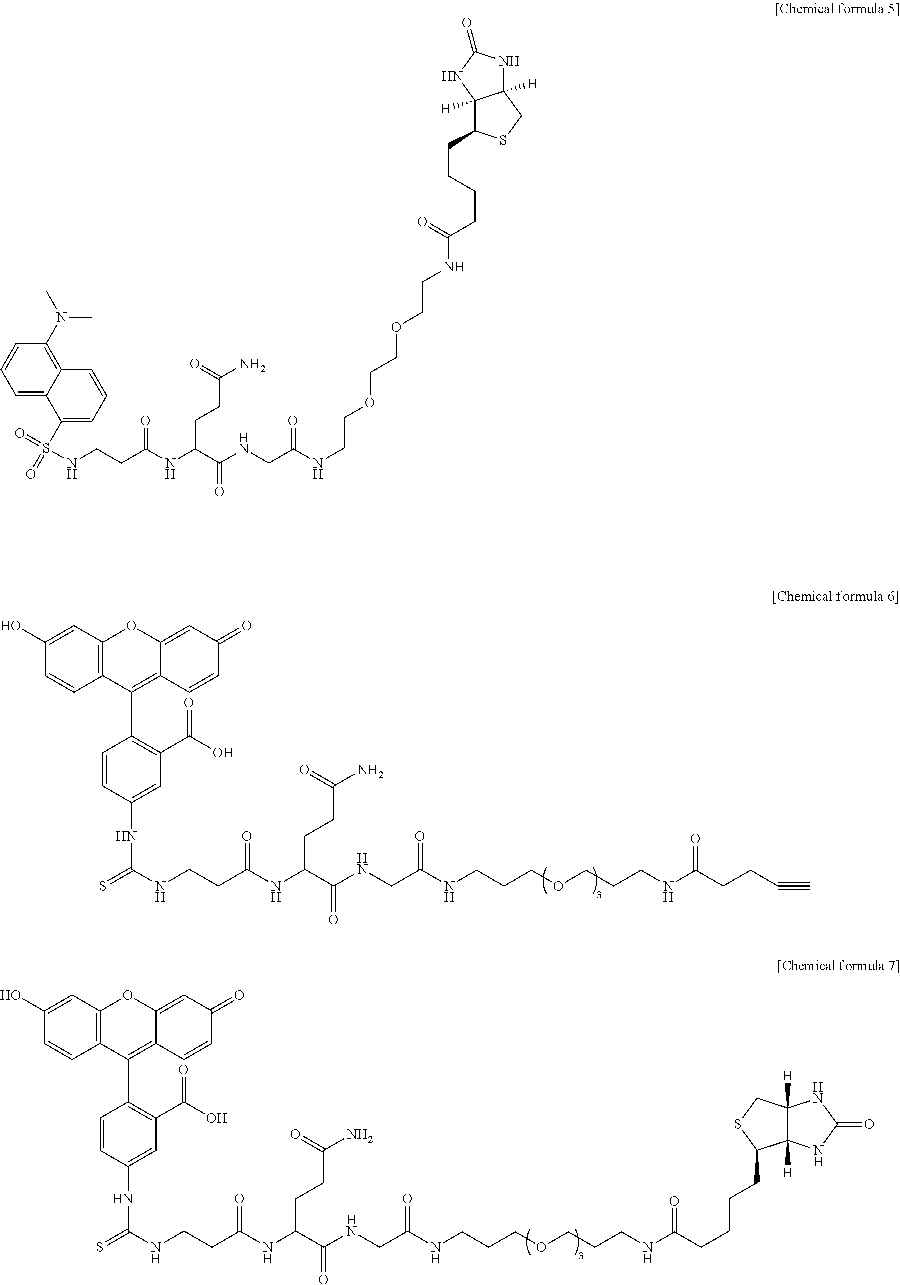

4) A fluorescently labeled protein obtained by TGase-catalyzed conjugating of a protein having a Lys residue capable of recognition by TGase and the substrate of TGase according to any one of 1) to 3).
5) The fluorescently labeled protein according to 4) wherein the protein is an IgG antibody.
6) A process for producing a fluorescently labeled protein which comprises the step of TGase-catalyzed conjugating of a protein having a Lys residue capable of recognition by TGase and the substrate of TGase according to any one of 1) to 3) to obtain a fluorescently labeled protein.
7) The process according to 6) wherein the protein is an IgG antibody.
8) A method for fluorescent labeling of a protein which comprises a step in which a functional protein having a Lys residue capable of recognition by TGase and a fluorescent group to which a portion having a Gln residue capable of recognition by TGase is bound via a linker are conjugated by TGase catalysis to obtain a fluorescently labeled functional protein with the function thereof left intact.
9) A method of labeling a protein which comprises the step of TGase-catalyzed conjugating of a protein having a Lys residue capable of recognition by TGase and a substrate of TGase represented by (fluorescent group)-(linker)-(a portion containing a Gln residue capable of recognition by TGase)-(functional organic molecule)
   (wherein:

the fluorescent group is fluorescein isothiocyanate (FITC), Texas Red (FE) or dansyl (Dns) or a group derived therefrom;

the linker is a group represented by —NH—(CH₂)ₙ—CO— (n is an integer of 1 to 6); and

the portion containing a Gln residue capable of recognition by TGase is QX (X is an amino acid residue other than Lys).
10) The method according to 9) wherein the portion containing a Gln residue capable of recognition by TGase is QG and the functional organic molecule is a group derived from biotin or alkyne.
11) The method according to 10) wherein the substrate of TGase is

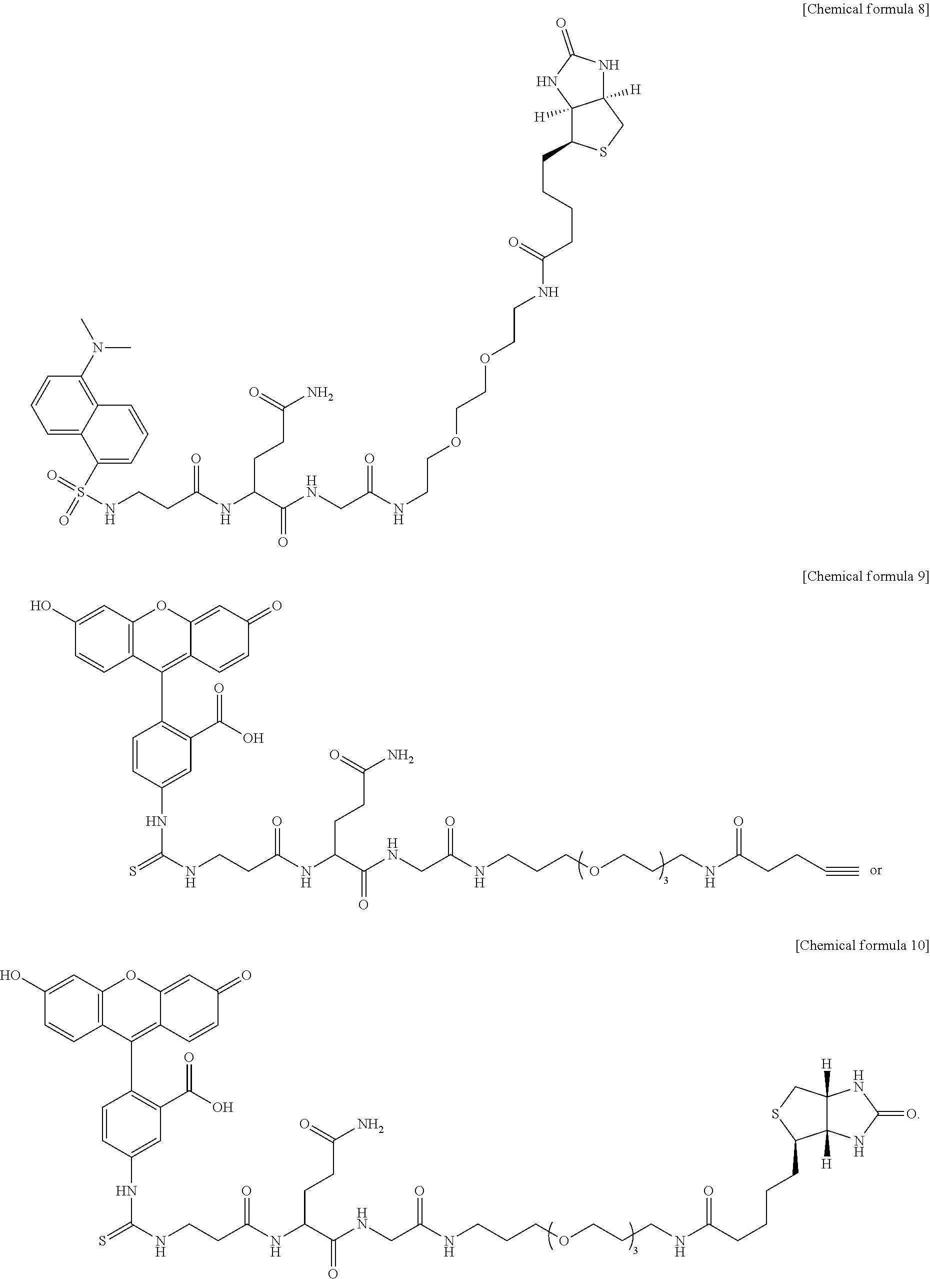

### BRIEF DESCRIPTION OF THE DRAWINGS

FIG. 1 shows the reactivity of MTG for FITC-ε-Acp-QG by the results of SDS-PAGE (left panel) and observation with a fluorescent imager (right panel).

FIG. 2 shows the reactivity of MTG for FITC-β-Ala-QG by the results of SDS-PAGE (left panel) and observation with a fluorescent imager (right panel).

FIG. 3 shows the reactivity of MTG for Flc-QG by the results of SDS-PAGE (left panel) and observation with a fluorescent imager (right panel).

FIG. 4 shows the reactivity of MTG for TR-β-Ala-QG by the results of SDS-PAGE (left panel) and observation with a fluorescent imager (right panel).

FIG. 5 shows the reactivity of MTG for Dns-ε-Acp-QG by the results of SDS-PAGE (left panel) and observation with a fluorescent imager (right panel).

FIG. 6 shows the result of evaluating the relative activity of NK6-AP with or without MTG-catalyzed modification.

FIG. 7 shows a time-dependent change in the MTG-catalyzed reaction of NK6-AP with FITC-β-Ala-QG.

FIG. 8 shows a change of substrate concentration in the MTG-catalyzed reaction of NK6-AP with FITC-β-Ala-QG.

FIG. 9 shows the results of TGase-catalyzed introduction of a fluorescent substrate into β-casein by SDS-PAGE (left panel) and by observation with a fluorescent imager (right panel).

FIG. 10 shows the intensity of an MTG-catalyzed reaction of an antibody with FITC-ε-Acp-QG by SDS-PAGE (left panel) and observation with a fluorescent imager (right panel).

FIG. 11 shows the intensity of an MTG-catalyzed reaction of an antibody with FITC-β-Ala-QG by SDS-PAGE (left panel) and observation with a fluorescent imager (right panel).

FIG. 12 shows the intensity of an MTG-catalyzed reaction of an antibody with TR-β-Ala-QG by SDS-PAGE (left panel) and observation with a fluorescent imager (right panel).

FIG. 13 shows the intensity of an MTG-catalyzed reaction of an antibody with Dns-ε-Acp-QG by SDS-PAGE (left panel) and observation with a fluorescent imager (right panel).

FIG. 14 shows the result of verification of antigen recognizing ability by ELISA for FITC-ε-Acp-QG (panel I) and FITC-β-Ala-QG (panel II).

FIG. 15 is a comparison for the intensity of fluorescence from AP.

FIG. 16 shows the results of observation in ELISA by a fluorescent imager (left panel) and fluorescent intensity data (right panel).

FIG. 17 shows the result of MALDI TOF-MS on Dns-β-Ala-QG-biotin after purification by HPLC.

FIG. 18 shows the results of verification by SDS-PAGE (left panel) and a transilluminator (right panel).

FIG. 19 shows the activity of NK6-AP before and after MTG-catalyzed introduction of Dns-β-Ala-QG-biotin.

FIG. 20 shows the result of measuring the absorbance of a dual-labeled protein after purification.

FIG. 21 shows the results of measuring the dansyl-derived fluorescent intensity of a dual-labeled protein immobilized on an avidin-coated microplate (left panel) and measuring the relative activity of AP (right panel).

FIG. 22 shows the result of MALDI TOF-MS after purification by HPLC.

FIG. 23 shows optimized pH conditions by the results of SDS-PAGE analysis (left panel) and fluorescent intensity analysis (right panel).

FIG. 24 shows optimized MTG concentrations by the results of SDS-PAGE analysis (left panel) and fluorescent intensity analysis (right panel).

FIG. 25 shows the reactivity of MTG for FITC-β-Ala-QG-alkyne by the result of SDS-PAGE analysis.

FIG. 26 shows the relative activity of NK6-AP before and after introduction of FITC-β-Ala-QG-alkyne.

FIG. 27 shows the result of conjugation between proteins through a click reaction by SDS-PAGE (left panel) and observation with a fluorescent imager (right panel).

FIG. 28 shows optimized pH conditions by the results of SDS-PAGE analysis (a band for the H chain of an antibody) (left panels) and fluorescent intensity analysis (right panel).

FIG. 29 shows optimized MTG concentrations by the results of SDS-PAGE analysis (a band for the H chain of an antibody) (left panels) and fluorescent intensity analysis (right panel).

FIG. 30 shows optimized substrate concentrations by the results of SDS-PAGE analysis (a band for the H chain of an antibody) (left panels) and fluorescent intensity analysis (right panel).

FIG. 31 shows optimized reaction temperatures by the result of SDS-PAGE analysis (panel row A) and a graph showing a time-dependent change on a fluorescent intensity basis (panel B).

FIG. 32 shows the result of immobilizing a multi-labeled antibody on an avidin-coated resin by SDS-PAGE analysis (CBB staining (upper panel) and fluorescent image (lower panel)).

### MODES FOR CARRYING OUT THE INVENTION

I. Novel Fluorescent Substrate of TGase

According to the present invention, there is provided a novel fluorescent substrate of TGase having the following structure:
(fluorescent group)-(linker)-(a portion containing a glutamine (Gln) residue capable of recognition by TGase)-R

The term “fluorescent group” as used in the present invention means, except in a special case, a group that absorbs a certain wavelength of electromagnetic radiation (e.g., light or X-rays) and which is capable of reradiating the absorbed energy as a longer wavelength of radiation. Examples of the fluorescent group include fluorescein isothiocyanate (FITC), Texas Red (TR; sulforhodamine), dansyl (Dns; (5-dimethylamino)naphthalene-1-sulfonyl), carbocyanine (Cy3, Cy4, PE-Cy5), DOXYL (N-oxy-4,4-dimethyloxazolidine), PROXYL (N-oxyl-2,2,5,5-tetramethylpyrrolidine), TEMPO (N-oxyl-2,2,6,6-tetramethylpiperidine), umbelliferrone, dinitrophenyl, acridine, coumarin, erythrosin, rhodamine, tetramethylrhodamine, 7-nitrobenzo-2-oxa-1-diazole (NBD), and groups derived therefrom.

When antibodies are to be labeled in accordance with the present invention, preferred examples of the fluorescent group are groups derived from FITC, TR, and Dns.

In the present invention, the fluorescent group has conjugated thereto, via a linker, the portion containing a Gln residue capable of recognition by TGase.

The term “linker” as used in the present invention means, except in a special case, a group that is used to conjugate the fluorescent group to the portion containing a Gln residue capable of recognition by TGase so as to form a thermochemically or optochemically inactive conjugate. A specific type of the linker to be used can be selected by any skilled artisan appropriately by considering various factors including its length, molecular flexibility, and the ease of reaction to be performed for conjugating.

The linker may be exemplified by a group represented by —NH—(CH2)ₙ—CO— (n is an integer of 1 to 6) and more specific examples include groups derived from β-alanine (β-Ala) and ε-aminocaproic acid (ε-Acp).

The “portion containing a Gln residue capable of recognition by TGase” in the present invention is preferably a substrate peptide of the smallest possible size and, considering the possibility of Lys to undergo self-crosslinking, it is preferably QS (X is an amino acid residue other than Lys.) Examples of the amino acid residue other than Lys are glycine (Gly), alanine (Ala), phenylalanine (Phe), aspartic acid (Asp), glutamic acid (Glu), isoleucine (Ile), leucine (Leu), valine (Val), methionine (Met), cysteine (Cys), asparagine (Asn), proline (Pro), glutamine (Gln), tyrosine (Tyr), serine (Ser), threonine (Thr), tryptophan (Trp), histidine (His), and arginine (Arg) residues. From the viewpoint of the capability of recognition by more than one TGase, the portion containing a Gln residue capable of recognition by TGase is preferably present as an L-glutamylglycyl group (QG).

Known as good substrates of microbial TGase are peptides composed of the amino acid sequences LLQG (SEQ ID NO: 1), LAQG (SEQ ID NO: 2), LGQG (SEQ ID NO: 3), PLAQSH (SEQ ID NO: 4), FERQHMDS (SEQ ID NO: 5) and TEQKLISEEDL (SEQ ID NO: 6), as well as peptides composed of the amino acid sequences GLGQGGG (SEQ ID NO: 7), GFGQGGG (SEQ ID NO: 8), GVGQGGG (SEQ ID NO: 9), and GGLQGGG (SEQ ID NO: 10). Also known as good substrates of guinea pig derived TGase are carbobenzoyl-L-glutamylphenylalanine (Z-QF), carbobenzoyl-L-glutamylglutamylglycine (Z-QQG), a peptide composed of the amino acid sequence EAQQIVM (SEQ ID NO: 11), as well as peptides composed of the amino acid sequences GGGQLGG (SEQ ID NO: 12), GGGQVGG (SEQ ID NO: 13), GGGQRGG (SEQ ID NO: 14), GQQQLG (SEQ ID NO: 15), PNPQLPF (SEQ ID NO: 16), and PKPQQFM (SEQ ID NO: 17). Depending on the type of TGase to be used, the portion containing a Gln residue capable of recognition by TGase may be present as the peptides mentioned above or as groups derived from such peptides.

Concerning the peptide referred to in the present invention, the term “a group derived” from it means a group that is formed by removing a hydrogen atom and/or a hydroxyl group from the N terminal and/or C terminal of that peptide.

The binding of a marker substance (e.g., the fluorescent group), the linker and the portion containing a Gln residue capable of recognition by TGase can be performed appropriately by any skilled artisan using various techniques that are implemented for similar purposes.

For example, the desired synthesis can be performed by the Fmoc or Boc method starting from a wang resin or Boc-Gly-PAM resin using a fully automatic solid-phase synthesizer of ABI 433A model. The Fmoc-amino acid, Fmoc-ε-Acp Boc-amino acid and Boc-β-Ala to be used may be of commercial grade. For more detailed conditions, see the Examples set forth in the present specification.

The moiety R may be a hydroxyl group or a functional organic molecule (an organic molecule having a particular function). An exemplary functional polymer is either a labeling substance for labeling a target substance or a group for immobilizing the target substance on a carrier or for supplying a reactive site to the target substance.

The term “labeling substance” as used in the present invention refers to a substance having a detectable functional group moiety either by itself or as part of a system for detection. Examples of the detectable functional group moiety include a fluorescent group (to be described later), digoxigenin, a dinitrophenyl group, a group containing a radioisotope, MRI contrast medium, an enzyme (e.g., alkaline phophatase, peroxidase, (-galactosidase, or glucose oxidase), biotin, a chemiluminescent group (a marker that can be detected upon light emission during chemical reaction), and an antibody.

The term “alkyne” as used in the present specification means, except in a special case, lower alkynes having 2 to 6 carbon atoms; a preferred example is a terminal alkyne (with one end bound to a hydrogen atom) and a more preferred example is a C₂ alkyne (propylene or methylacetylene).

In order to ensure that the protein as the target to be labeled can be labeled while maintaining its function, it is sometimes preferred that the functional organic molecule is a comparatively small molecule. It is also sometimes preferred that the functional organic molecule has high water solubility because the use of organic solvents that might adversely affect the three-dimensional structure of the protein can be reduced or entirely eliminated. The smallness of the molecular weight also benefits the administration to cells.

Examples of R that are preferred in the present invention are groups derived from biotins (more specifically an aminated biotin), alkynes, maleimide, nucleic acids, azides or cyclopentadiene.

The term “groups derived” from biotins, nucleic acids, polyethylene glycol, azides, alkynes, maleimide or cyclopentadiene as used in the present invention refers to groups that are derived from these substances, with an optionally added spacer portion (polyethylene oxide (PEO)ₙ (also known as polyethylene glycol (PEG)ₙ); n is an integer of 1 to 6, say, 2 or 3), and which can be conjugated to the C terminal of the portion having a Gln residue capable of recognition by TGase (and which may be exemplified by a group formed by removing a hydrogen atom, a hydroxyl group or the like from the side chain).

If R is a functional organic molecule, it is advantageously a group having a suitable length of spacer in order to reduce steric hindrance. Although an aminated biotin having a suitable length of spacer is occasionally referred to simply as “biotin” in the present specification, the context will tell which is the proper interpretation. This also applies to alkynes having a suitable length of spacer.

In a particularly preferred embodiment of the present invention, biotin (aminated biotin having a spacer) can be used as R. The following aminated biotin (commercially available under the trade name EZ-Link (registered trademark) Amine-PEO₂-Biotin (PIERCE) is this preferred example:

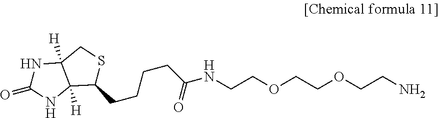

A substrate of the present invention in which R is biotin can be synthesized by any skilled artisan according to a procedure of Fmoc solid-phase peptide synthesis. For more details of the procedure and conditions for synthesis, reference can be made to Examples of the present invention. The substrate of the present invention in which R is biotin can, after being conjugated to a protein as catalyzed by TGase, be immobilized on an avidin-coated solid phase.

In a particularly preferred embodiment of the present invention, R may be an alkyne which is a reaction site for the Huisgen cycloaddition, a typical reaction in the click chemistry. Click reactions involve very convenient experimental procedures and are capable of proceeding with high efficiency in water without yielding any byproducts. For the click reactions, reference can be made to such articles as P. C. Lin, S. H. Ueng, M. C. Tseng, J. L. Ko, K. T. Huang, S. C. Yu, A. K. Adak, Y. J. Chen, C. C. Lin, Angew. Chem. Int. Ed., 2006, 45, 4286-4290; T. S. Seo, Z. Li, H. Ruparel, J. Ju, J. Org. Chem., 2003, 68, 609-612; and T. Govindaraju, P. Jonkheijm, L. Gogolin, H. Schroeder, C. F. W. Becker, C. M. Niemeyer, H. Waldmann, Chem. Comm., 2008, 3723-3725.

In the present invention, R can be designed as a reaction site for click reactions. Among click reactions, the azide alkyne Huisgen cycloaddition in the absence of a catalyst or using a copper catalyst is one of the most preferred examples that can be applied in the present invention.

A substrate of the present invention in which R is an alkyne can be synthesized by any skilled artisan according to a procedure of Fmoc solid-phase peptide synthesis using a commercial resin (say, Universal-PEG NovaTag^{TR} Resin). For more details of the procedure and conditions for synthesis, reference can be made to Examples of the present invention. The substrate of the present invention in which R is an alkyne can, after being conjugated to a protein as catalyzed by TGase, be further conjugated to an azide form of another protein by a click reaction.

According to the present invention, a novel fluorescent substrate of transglutaminase is provided. This fluorescent substrate is useful as a reagent for fluorescent labeling of recombinant proteins or a reagent for fluorescent labeling of antibodies (in a kit) or for the purpose of preparing protein arrays or antibody arrays through site-specific immobilization or for molecular imaging and drug targeting that rely on fluorescently labeled antibodies.

II. Proteins as the Target of Labeling

Proteins to be used in the method of the present invention as the target of labeling are either proteins that have a Lys group capable of recognition by TGase or proteins that have added to the C or N terminal a peptide containing a Lys group capable of recognition by TGase.

Examples of the proteins that have a Lys group capable of recognition by TGase include IgG antibodies and casein.

Examples of peptides containing a Lys group capable of recognition by TGase include MKHKGS (SEQ ID NO: 18), modified S-peptide (GSGMKETAAARFERAHMDSGS (SEQ ID NO: 19)), MGGSTKHKIPGGS (SEQ ID NO: 20), N-terminal glycine (N-terminal GGG, N-terminal GGGGG (SEQ ID NO: 21)), and MKHKGSGGGSGGGS (SEQ ID NO: 22) with an extended linker site between the N-terminal MKHKGS and the target protein.

The proteins that have added thereto the peptide containing a Lys group capable of recognition by TGase are preferably proteins that can be produced by genetic engineering techniques since they can be prepared as recombinant proteins. Examples of such proteins include alkaline phosphatase (AP), green fluorescent protein (GFP), glutathione-S-transferase (GST), luciferase, peroxidase, and peptides containing antibody epitope sequences.

The recombinant proteins may further have (His)₆-tag added to the N or C terminal for purification. To circumvent a drop in the reactivity of TGase, a preferred design is such that His-tag is introduced at a terminal different from the one at which a substrate peptide tag has been introduced. While such proteins can be appropriately prepared by any skilled artisan using various techniques that are implemented for similar purposes, an alkaline phosphatase (NK6-AP) with the MKHKGS sequence containing a TGase active Lys residue being present at the N-terminal can be prepared by referring to the following procedure. Briefly, an amplification vector containing an AP encoding DNA is obtained or prepared and the AP encoding DNA is amplified by PCR; in this case, primers are designed such that a site (restriction site) that can be recognized by a suitable restriction enzyme and an MKHKGS encoding DNA are introduced on the N-terminal side whereas a suitable restriction enzyme and an optional His-tag encoding DNA are introduced on the C-terminal side. After amplification, the resulting DNA is treated with restriction enzymes and inserted into an expression vector similarly treated with restriction enzymes, whereby an NK6-AP expressing vector is prepared. The resulting expression vector is introduced into a suitable host (e.g., E. coli) to transform it; the resulting transformant is cultured in a selection medium. Recombinant AP containing fractions from the culture are purified on a His-tag and/or gel filtration column to prepare a recombinant AP. The recombinant AP may optionally be checked for its sequence or purity by a suitable means.

III. Transglutaminase-Catalyzed Reaction

In the present invention, various kinds of transglutaminase can be used. Currently known TGase's are those derived from mammals (guinea pig and human), invertebrates (insect, horseshoe crab, and sea urchin), plants, fungi, and protozoans (slime mold); in humans, eight kinds of enzymes have been discovered. Any skilled artisan may refer to the examples specifically disclosed in the present invention and set appropriate conditions considering the substrate specificities of the respective TGase's and other factors to determine which one of those TGase's can be used. Preferred examples of TGase are microbial ones (MTG) and those which are derived from guinea pig liver (GTG).

In the method of the present invention, transglutaminase-catalyzed reactions can be carried out under mild conditions, for example, at 4° C. for several hours to one day. Under the conditions applied by the present inventors, an MTG-catalyzed reaction between NK6-AP and FITC-β-Ala-QG ended in approximately 3 hours (see Examples).

In the present invention, even if the concentration of a substrate containing a labeling substance (say, a fluorescent substrate) is relatively low, reactions will proceed to achieve sufficient labeling to allow for detection. Under the conditions applied by the present inventors, labeling reaction proceeded even when the final concentration was dropped to 1 μM (see Examples).

IV. Others (Effects, Uses, Utility, etc. of the Present Invention

According to the present invention, it was verified that the novel fluorescent substrates, FITC-ε-Acp-QG, FITC-β-Ala-QG, TR-β-Ala-QG, and Dns-β-Acp-QG, served as substrates of MTG. By using these substrates, proteins can be labeled in a site-specific manner. The proteins labeled at appropriate sites will not undergo a loss in their function from the labeling.

The present invention also enables the labeling of antibodies, in which case the labeling is performed on H chains (heavy chains). Again, the labeling of an antibody will not cause a loss in its antigen recognizing ability.

Fluorescently labeled antibodies that are obtained by the present invention are useful in ELISA (enzyme-linked immunosorbent assay), immunostaining, and flow cytometry.

According to the present invention, a solution containing both a fluorescent substrate and a protein to be labeled simply needs the addition of transglutaminase to effect fluorescent labeling.

A fluorescent substrate of the present invention which utilizes a peptide (a good substrate of TGase) as the portion containing a Gln residue capable of recognition by TGase may take the advantage of the free C-terminal of the peptide to impart additional functions. For example, biotin, DNA, magnetic nanoparticles, or azides, alkynes, maleimide, cyclopentadiene derivatives, etc. may be bound to enable dual labeling.

For example, a dual labeled substrate having a linker and a fluorescent group introduced on the N-terminal side of Gln and an aminated biotin introduced at the C-terminal of Gly has high detection sensitivity, provides a wide dynamic range of quantification, and also has a potential for imaging applications. Since biotin has an extremely high affinity for avidin, the interaction between biotin and avidin may be used as a mediator to immobilize the protein on microplates, quantum dots, fluorescent nanoparticles, fine magnetic particles, etc. while retaining its function. As the result of immobilization, the background can be reduced and the detection sensitivity improved. As a further advantage, the profile of activity vs. immobilization yield can be plotted to calculate the specific activity of an enzyme; this is expected to contribute to the development of protein arrays by encouraging the quantitative discussion that has been a bottleneck in the art.

For example, a substrate of the present invention that has a linker and a fluorescent group introduced on the N-terminal side of Gln and an alkylene introduced at the C-terminal of Gly is a peptide having a site-specific reaction site and a covalently bonding reaction site and enables conjugation of dissimilar proteins.

### EXAMPLES

### Example 1

### Synthesis of Fluorescent Substrates

The model of a fluorescent group was Z-QG known as a good substrate of MTG and the bulky and hydrophobic N-benzyloxycarbonyl group was replaced by fluorescein, Texas Red, and dansyl. Either no linker was used or ε-Acp (aminocaproic acid) or β-Ala was selected as a linker.

1. Synthesis of FITC-ε-Acp-QG

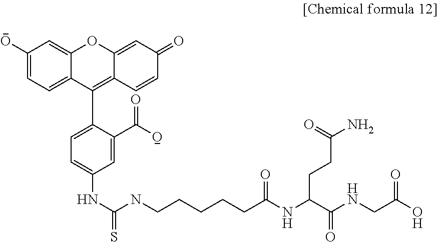

A peptide resin was synthesized by the Fmoc method starting with a wang resin (product of WATANABE CHEMICAL INDUSTRIES, LTD.) and using a fully automatic solid-phase synthesizer of ABI 433A model. Fmoc-Gly and Fmoc-Gln (Trt) (products of PEPTIDE INSTITUTE INC.), Fmoc-ε-Acp (product of WATANABE CHEMICAL INDUSTRIES, LTD.) and fluorescein-4-isothiocyanate (product of DOJINDO) were used.

The resulting peptide resin was treated with TFA/H₂O/triisopropylsilane (95:5:5) at room temperature for 1.5 hours and sliced to obtain a crude peptide.

The resulting crude peptide was purified by gradient elution on a reverse-phase HPLC column (ODS) using a 0.1% TFA containing H₂O—CH₃CN system. Fractions containing the end product in high purity were collected and freeze-dried to yield a yellow sample of FITC-ε-Acp-QG (purity: 96.7%).

Result of amino acid analysis (6N HCl, 110° C., 22 hrs): Gln(1) 1.01, Gly(l) 1.00, NH₂(1) 1.03, ε-Acp(1) 1.00;

ESI-MS: MH⁺=706.3 (Theor. 706.2, monoisotopic).

2. Synthesis of FITC-β-Ala-QG

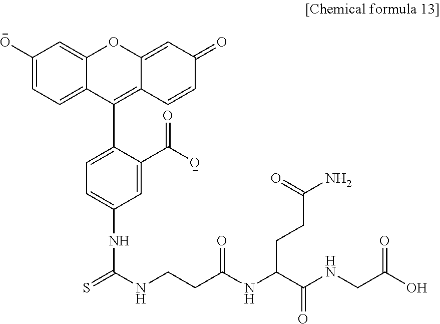

A peptide resin was synthesized by the Boc method starting with a Boc-Gly-PAM resin (product of BeadTech Inc.) and using a fully automatic solid-phase synthesizer of ABI 430A model. Boc-Gln and Boc-β-Ala (products of PEPTIDE INSTITUTE INC.) and fluorescein-4-isothiocyanate (product of DOJINDO) were used.

The resulting peptide resin was treated with anhydrous hydrogen fluoride/p-cresol (8:2) at −5 to −2° C. for an hour and sliced to obtain a crude peptide.

The resulting crude peptide was purified by gradient elution on a reverse-phase HPLC column (ODS) using a 0.1% TFA containing H₂O—CH₃CN system. Fractions containing the end product in high purity were collected and freeze-dried to yield a yellow sample of FITC-β-Ala-QG (purity: 98.8%).

Result of amino acid analysis (6N HCl, 110° C., 22 hrs): Gln(1) 1.01, Gly(l) 1.00, NH₂(1) 1.07, β-Ala(1) 1.06;

ESI-MS: MH⁺=664.2 (Theor. 664.2, monoisotopic).

3. Synthesis of TR-ε-Acp-QG

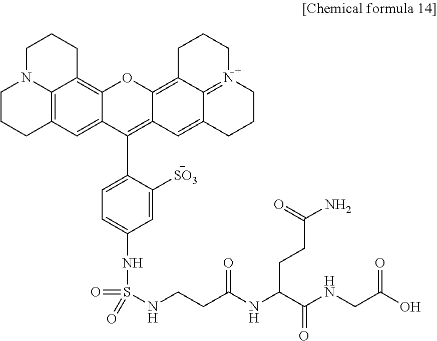

An ε-Acp-QG peptide resin was synthesized by the Fmoc method starting with a wang resin (product of WATANABE CHEMICAL INDUSTRIES, LTD.) and using a fully automatic solid-phase synthesizer of ABI 433A model. Fmoc-Gly and Fmoc-Gln (Trt) (products of PEPTIDE INSTITUTE INC.) and Fmoc-ε-Acp (product of WATANABE CHEMICAL INDUSTRIES, LTD.) were used.

The resulting peptide resin was treated with TFA/H₂O/triisopropylsilane (95:5:5) at room temperature for 1.5 hours and sliced to obtain a crude ε-Acp-QG peptide. The crude peptide was reacted with Sulforhodamine 101 acid chloride (product of DOJINDO) to yield a crude form of the desired TR-ε-Acp-QG peptide.

The resulting crude peptide was purified by gradient elution on a reverse-phase HPLC column (ODS) using a 0.1% TFA containing H₂O—CH₃CN system. Fractions containing the end product in high purity were collected and freeze-dried to yield a red purple sample of TR-ε-Acp-QG (purity: 98.1%).

Result of amino acid analysis (6N HCl, 110° C., 22 hrs): Gln(1) 1.00, Gly(l) 0.99, NH₂(1) 1.02, 13-Ala(1) 0.99

ESI-MS: (M−H)⁻ 861.3 (theoretical 861.3, monoisotopic).

4. Synthesis of Dns-ε-Acp-QG

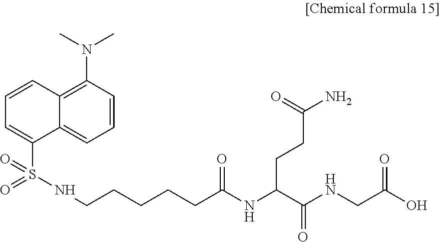

A peptide resin was synthesized by the Fmoc method starting with a wang resin (product of WATANABE CHEMICAL INDUSTRIES, LTD.) and using a fully automatic solid-phase synthesizer of ABI 433A model. Fmoc-Gly and Fmoc-Gln (Trt) (products of PEPTIDE INSTITUTE INC.), Fmoc-ε-Acp (product of WATANABE CHEMICAL INDUSTRIES, LTD.) and dansyl chloride (product of Tokyo Chemical Industry Co., Ltd.) were used.

The resulting peptide resin was treated with TFA/H₂O/triisopropylsilane (95:5:5) at room temperature for 1.5 hours and sliced to obtain a crude peptide.

The resulting crude peptide was purified by gradient elution on a reverse-phase HPLC column (ODS) using a 0.1% TFA containing H₂O—CH₃CN system. Fractions containing the end product in high purity were collected and freeze-dried to yield a pale yellow sample of Dns-ε-Acp-QG (purity: 98.2%).

Result of amino acid analysis (6N HCl, 110° C., 22 hrs): Gln(1) 1.01, Gly(l) 1.00, NH₂(1) 1.09, ε-Acp(1) 0.18 (under this condition, Dns-ε-Acp is considerably less likely to be cut);

ESI-MS: MW=549.3 (Theor. 549.6).

5. Synthesis of Flc-QG

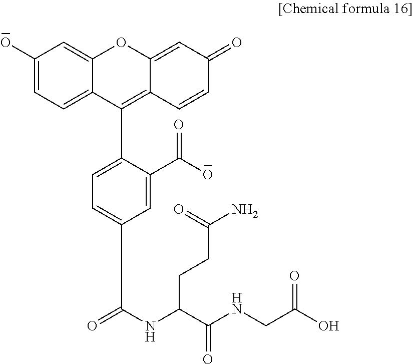

A peptide resin was synthesized by the Fmoc method starting with a wang resin (product of WATANABE CHEMICAL INDUSTRIES, LTD.) and using a fully automatic solid-phase synthesizer of ABI 433A model. Fmoc-Gly and Fmoc-Gln (Trt) (products of PEPTIDE INSTITUTE INC.) and 5-carboxyfluorescein (product of Toronto Research Chemicals Inc.) were used.

The resulting peptide resin was treated with TFA/H₂O/triisopropylsilane (95:5:5) at room temperature for 1.5 hours and sliced to obtain a crude peptide.

The resulting crude peptide was purified by gradient elution on a reverse-phase HPLC column (ODS) using a 0.1 M AcONH₄ containing H₂O—CH₃CN system. Fractions containing the end product in high purity were collected and freeze-dried to yield an orange sample of Flc-QG (purity: 99.2%).

Result of amino acid analysis (6N HCl, 110° C., 22 hrs): Gln(1) 1.02, Gly(l) 1.00, NH₂(1) 1.66;

ESI-MS: MW=561.32 (theoretical 561.2).

### Example 2

### Reactivity of Fluorescent Substrates with Proteins in MTG-Catalyzed Reaction

1. Introduction

In this Example, a study was made to see whether a Gln-containing fluorescent substrate and an enzyme into which a Lys tag was introduced by a genetic engineering technique would be modified in a site-specific manner; after it was verified that the fluorescent substrate was a substrate recognizable by MTG, the effect of the introduction on the enzymatic activity was investigated. In addition, to show the independency of the fluorescent substrate on the origin of TGase that would recognize it, a study was also made to evaluate the reactivity of the fluorescent substrate for GTG, a TGase derived from the guinea pig liver.

An alkaline phosphatase (AP) a native type of which is known to be incapable of acting as a substrate of MTG was selected as a model protein.

It is known that AP, when a Lys tag is introduced at the N terminal by a genetic engineering technique, becomes a good substrate of MTG (see NK6-AP in the table below) and in this Example, NK6-AP was used as a substrate on the Lys side to evaluate the reactivity of the fluorescent substrate.

TABLE 1

| | Amino acid sequence of |
|---|---|
| Enzyme | N- and C-terminal region |
| WT-AP | TPEMP-ALGLK |
| NK6-AP | MKHKGSTPEMP-ALGLKLEHHHHHH |

2. Experiment

2-1. Reagents

MTG was kindly supplied from AJINOMOTO. NK6-AP was prepared by the previously reported method, with necessary modifications. For the method of producing NK6-AP, reference can be made not only to the disclosures in the specification of the subject application but also to Patent Document 1, supra. The fluorescent substrates synthesized in Example 1 were used in the experiment. The other reagents were commercially available.

2-2. MTG-Catalyzed Introduction of Fluorescent Substrates into NK6-AP

To determine whether the respective fluorescent substrates would serve as substrates of MTG, a study was made to see whether the Gln-containing fluorescent substrates and AP (NK6-AP) into which a Lys tag was introduced by a genetic engineering technique would be modified in a site-specific manner.

To perform a binding reaction, the fluorescent substrate (FITC-ε-Acp-QG, FITC-β-Ala-QG, Flc-QG, TR-β-Ala-QG or Dns-ε-Acp-QG), NK6-AP and MTG were added to 10 mM Tris-HCl (pH 8) to give respective final concentrations of 1 mM, 0.5 mg/ml and 1 U/ml and they were thoroughly agitated and the resulting solution was left to stand overnight at 4° C. For the purpose of comparison, an MTG-free sample was prepared.

After the reaction, the samples were subjected to SDS-polyacrylamide gel electrophoresis (SDS-PAGE). The gel from SDS-PAGE was examined with a fluorescent imager before it was stained with Coomassie Brilliant Blue (CBB) in solution. For the sample containing Dns-ε-Acp-QG, examination was made with a transilluminator.

2-3. Evaluating the Effect of Modification on the Enzymatic Activity of NK6-AP

The effect of introduction on the enzymatic activity was studied. In the study, p-nitrophenylphosphoric acid (p-NPP) was used as a substrate and the activities of the samples prepared in Experiment 2-2 were measured; AP would catalyze the dephosphorylation of p-NPP to form p-nitrophenol (p-NP). The reaction conditions for activity measurement were AP 28 nM, p-NPP 1 mM and pH 8.0 and at a controlled temperature of 27° C., the p-NP derived absorption at 410 nm was traced.

2-4. Time Course of MTG-Catalyzed Reaction of Fluorescent Substrate with NK6-AP

A study was made using FITC-β-Ala-QG as a fluorescent substrate. The reaction conditions for introducing FITC-β-Ala-QG into NK6-AP were as follows: FITC-β-Ala-QG, NK6-AP and MTG were added to 10 mM Tris-HCl (pH 8) to give respective final concentrations of 0.1 mM, 0.5 mg/ml and 0.1 U/ml and they were thoroughly agitated at 4° C. for 0-5 hours. To trace the binding, the gel from SDS-PAGE was analyzed by a fluorescent imager.

2-5. Substrate Concentration Dependency of MTG-Catalyzed Reaction of Fluorescent Substrate with NK6-AP

A study was made using FITC-β-Ala-QG as a fluorescent substrate. The reaction conditions for introducing FITC-β-Ala-QG into NK6-AP were as follows: FITC-β-Ala-QG, NK6-AP and MTG were added to 10 mM Tris-HCl (pH 8) to give respective final concentrations of 1-1000 μM, 0.5 mg/ml and 0.1 U/ml and they were thoroughly agitated at 4° C. for 15 minutes. Tracing on the sample after the reaction was performed by the same procedure as in Experiment 2-4.

2-6. Evaluating the Reactivity of GTG for Fluorescent Substrate

In the experiment, β-casein was used as a protein having a TGase-recognizable Lys and a reaction for crosslinking with a fluorescent substrate was performed to evaluate its reactivity for GTG.

To perform a binding reaction, FITC-β-Ala-QG, β-casein and GTG were added to 50 mM HEPES (5 mM NaCl, 10 mM CaCl₂, pH 7.4) to give respective final concentrations of 1 mM, 0.5 mg/ml and 0.2 U/ml and the resulting mixture was left to stand overnight at 27° C. Two comparative samples were prepared; one was a GTG-free sample and the other was prepared by performing an MTG-catalyzed reaction. For the MTG-catalyzed binding reaction, FITC-β-Ala-QG, β-casein and MTG were added to a 10 mM phosphate buffer (pH 7) to give respective final concentrations of 1 mM, 0.5 mg/ml and 1 U/ml and the resulting mixture was left to stand overnight at 27° C. Tracing on the sample after the reaction was performed by the same procedure as in Experiment 2-2.

3. Experimental Results and Discussion

3-1. Evaluating the Reactivity of MTG for Fluorescent Substrates

A Gln-containing fluorescent substrate was reacted with NK6-AP, which was known to become a good substrate of MTG when a Lys tag was introduced into it by a genetic engineering technique (see below) and after performing SDS-PAGE, the fluorescence from the resulting gel was observed for tracing purposes.

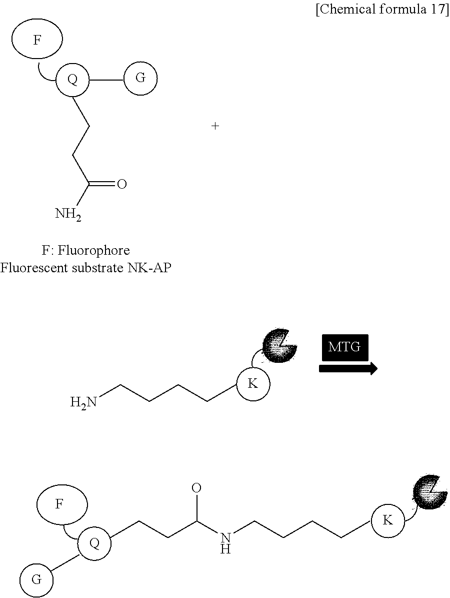

3-1-1. Reactivity of MTG for FITC-ε-Acp-QG

FIG. 1 shows the result of SDS-PAGE on samples after the reaction between FITC-ε-Acp-QG and NK6-AP, as well as the result of observing the gel from SDS-PAGE with a fluorescent imager. In SDS-PAGE, a band of NK6-AP was detected at ca. 45 kDa and a band of MTG at ca. 35 kDa. The fluorescent images revealed that FITC-derived fluorescence was observed at ca. 45 kDa in the lane to which MTG had been added but no fluorescence was observed in the lane to which no MTG had been added. Thus, it was verified that FITC-ε-Acp-QG is introduced into NK6-AP by the MTG-catalyzed reaction to become recognizable as a substrate of MTG.

3-1-2. Reactivity of MTG for FITC-β-Ala-QG

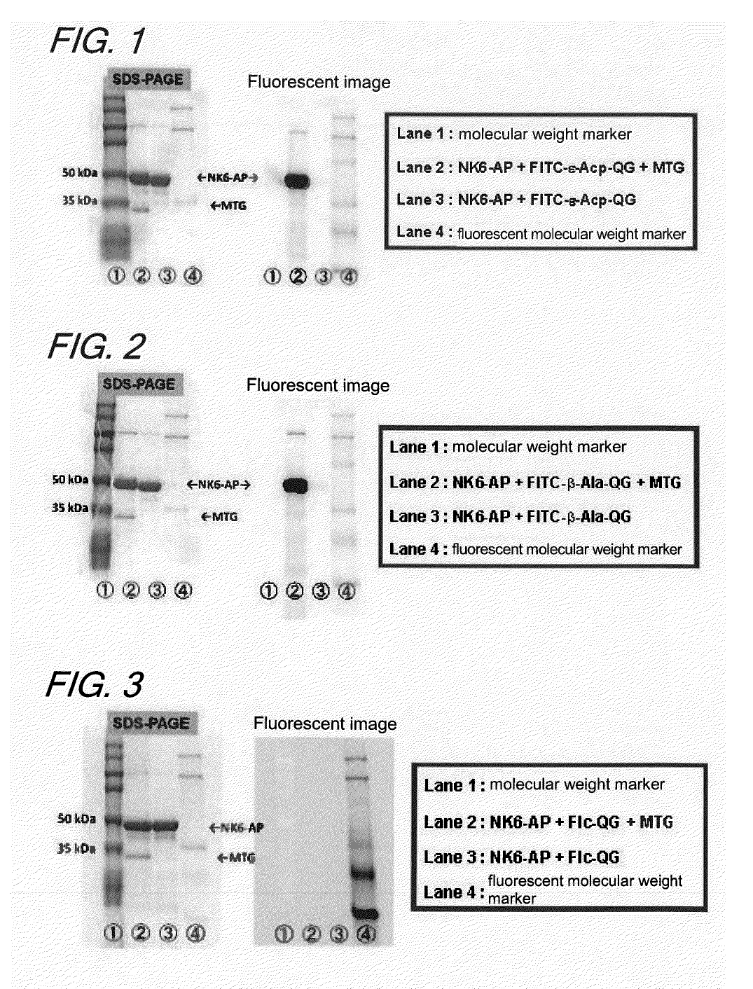

FIG. 2 shows the result of SDS-PAGE on samples after the reaction between FITC-β-Ala-QG and NK6-AP, as well as the result of observing the gel from SDS-PAGE with a fluorescent imager. In SDS-PAGE, a band of NK6-AP was detected at ca. 45 kDa and a band of MTG at ca. 35 kDa. The fluorescent images revealed that FITC-derived fluorescence was observed at ca. 45 kDa in the lane to which MTG had been added but no fluorescence was observed in the lane to which no MTG had been added. Thus, it was verified that FITC-β-Ala-QG is introduced into NK6-AP by the MTG-catalyzed reaction to become recognizable as a substrate of MTG.

3-1-3. Reactivity of MTG for Flc-QG

FIG. 3 shows the result of SDS-PAGE on samples after the reaction between Flc-QG and NK6-AP, as well as the result of observing the gel from SDS-PAGE with a fluorescent imager. In SDS-PAGE, a band of NK6-AP was detected at ca. 45 kDa and a band of MTG at ca. 35 kDa. The fluorescent images revealed no observation of fluorescence derived from fluorescein. Thus, it was verified that Flc-QG is not recognizable by MTG. In view of the recognizability of FITC-ε-Acp-QG and FITC-β-Ala-QG, a cause for the unrecognizability of Flc-QG would be steric hindrance to the recognization of Gln by MTG. It was therefore verified to be useful to introduce a linker at the N terminal of Gln.

3-1-4. Reactivity of MTG for TR-β-Ala-QG

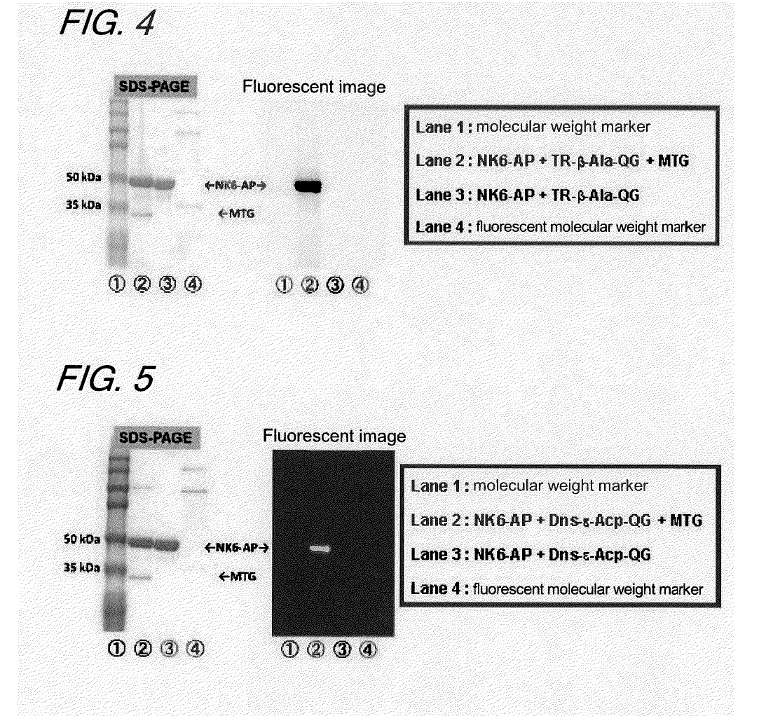

FIG. 4 shows the result of SDS-PAGE on samples after the reaction between TR-β-Ala-QG and NK6-AP, as well as the result of observing the gel from SDS-PAGE with a fluorescent imager. In SDS-PAGE, a band of NK6-AP was detected at ca. 45 kDa and a band of MTG at ca. 35 kDa. The fluorescent images revealed that fluorescence derived from Texas Red was observed at ca. 45 kDa in the lane to which MTG had been added but no fluorescence was observed in the lane to which no MTG had been added. Thus, it was verified that TR-β-Ala-QG is introduced into NK6-AP by the MTG-catalyzed reaction to become recognizable as a substrate of MTG.

3-1-5. Reactivity of MTG for Dns-ε-Acp-QG

FIG. 5 shows the result of SDS-PAGE on samples after the reaction between Dns-ε-Acp-QG and NK6-AP, as well as the result of observing the gel from SDS-PAGE with a transilluminator. In SDS-PAGE, a band of NK6-AP was detected at ca. 45 kDa and a band of MTG at ca. 35 kDa. The fluorescent images revealed that dansyl-derived fluorescence was observed at ca. 45 kDa in the lane to which MTG had been added but no fluorescence was observed in the lane to which no MTG had been added. Thus, it was verified that Dns-ε-Acp-QG is introduced into NK6-AP by the MTG-catalyzed reaction to become recognizable as a substrate of MTG.

3-2. Evaluating the Relative Activity of NK6-AP After Introduction

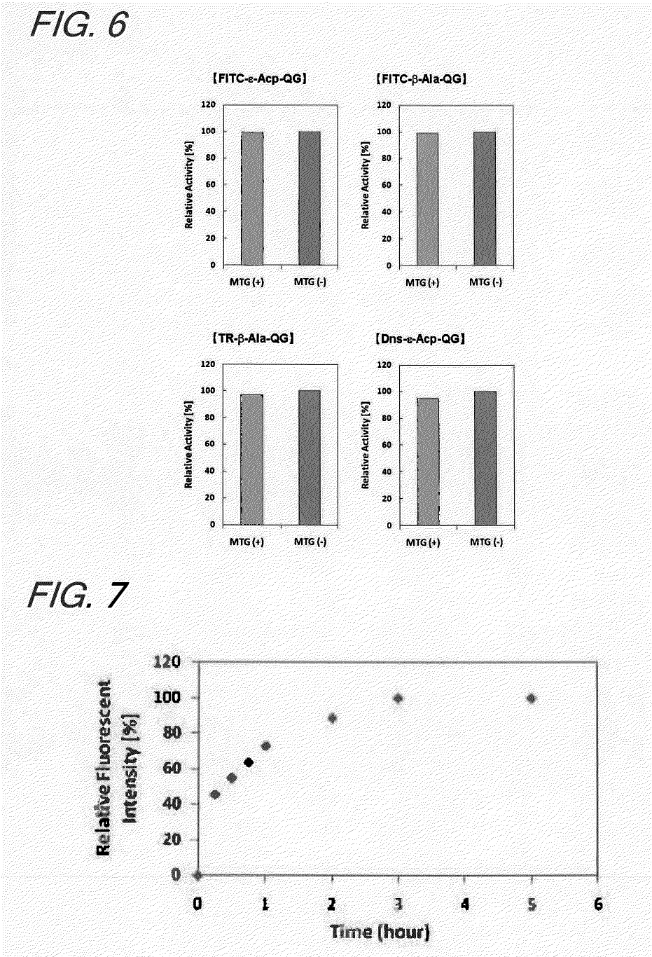

FIG. 6 shows the result of evaluating the relative activity of NK6-AP after introduction. As it turned out, the activity of AP hardly changed whether MTG was added or not. It was therefore suggested that a fluorescent substrate can be introduced into NK6-AP without causing a functional loss from the modification with MTG.

3-3. Time-Dependent Change in the MTG-Mediated Reaction of NK6-AP with Fluorescent Substrate

FIG. 7 shows a time-dependent change in the MTG-mediated reaction of NK6-AP with FITC-β-Ala-QG. It was clear from the result that the reaction virtually ended in about 3 hours under the conditions employed.

3-4. Substrate Concentration Dependency of the MTG-Mediated Reaction of NK6-AP with Fluorescent Substrate.

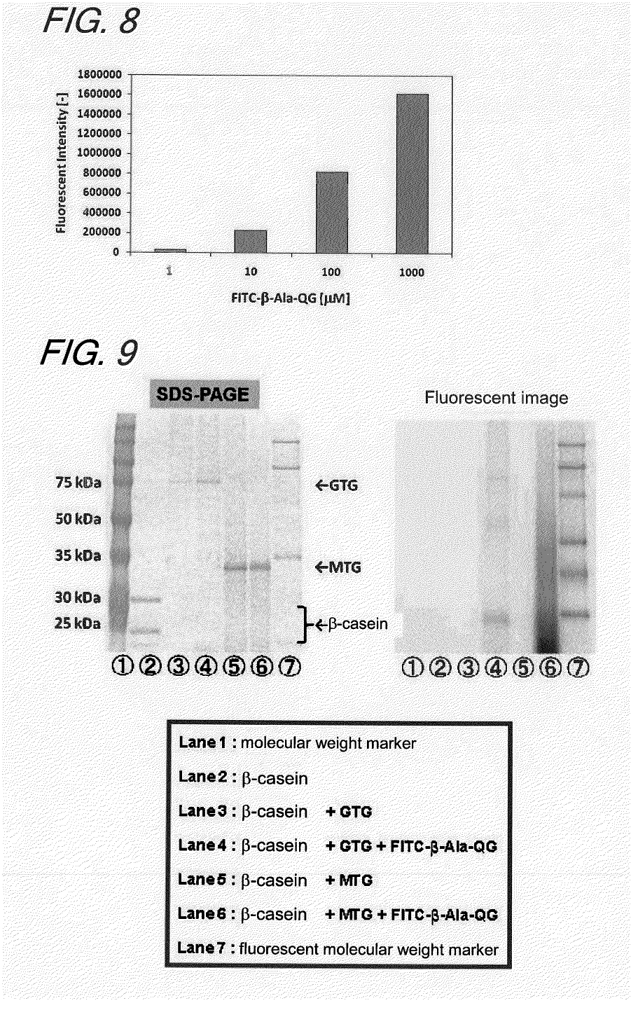

FIG. 8 shows a change of substrate concentration in the MTG-mediated reaction of NK6-AP with FITC-β-Ala-QG. The result confirmed that under the conditions employed, the reaction progressed and detection was possible even when the concentration of the substrate was reduced to 1 μM at a minimum level. The reaction also progressed at a maximum level of 1 mM. No experiment could be performed at higher concentrations because the proportion of the substrate dissolving DMSO in the aqueous solution would change.

3-5. Evaluating the Reactivity of Fluorescent Substrate for GTG

To study the reactivity of GTG (TGase derived from guinea pig liver) for a fluorescent substrate, an evaluation was made as to whether the fluorescent substrate would prove reactive in the reaction for the crosslinking of TGase in the presence of β-casein known as a good substrate of TGase.

Casein is a typical example of phosphoproteins in which phosphate groups are bound to many of the Ser residues among the amino acids that compose the proteins. Because of this feature, the casein molecule, seen as a whole, is negatively charged and easy to bind calcium or sodium ions. It is also known that casein does not have secondary structures such as α-helices and β-sheets in its molecular structure but has a plurality of Gln and Lys residues that are recognizable by TGase. Hence, β-casein will experience a reaction for self-crosslinking in the presence of TGase. If a fluorescent substrate having TGase recognizable Gln is added, the β-casein as it experiences a self-crosslinking reaction would incorporate the fluorescent substrate to cause a binding reaction according to the scheme depicted below. In the experiment under consideration, FITC-β-Ala-QG was used as the fluorescent substrate.

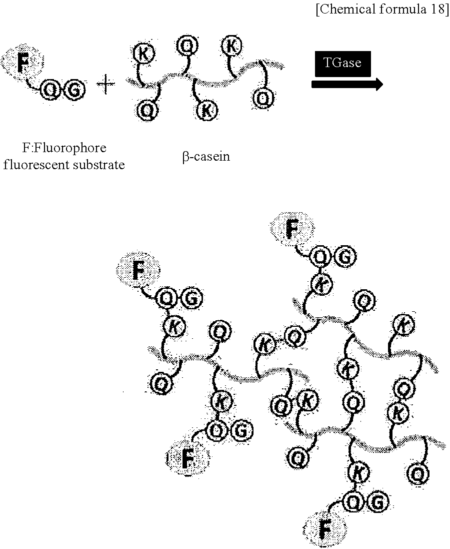

FIG. 9 shows the result of SDS-PAGE on samples after the reaction, as well as the result of examining the gel from SDS-PAGE with a fluorescent imager. The result of SDS-PAGE shows the loss of β-casein in both lanes to which GTG and MTG were added, indicating the occurrence of their crosslinking. The fluorescent images revealed that FITC-derived fluorescence was observed in the two lanes to which FITC-β-Ala-QG had been added together with GTG or MTG. Thus, it was verified that FITC-β-Ala-QG is also recognized by MTG. Of these two lanes, the one treated with MTG showed a higher fluorescence intensity, thus confirming the higher reactivity of MTG. This would be due to the difference in substrate specificity between MTG and GTG.

4. Conclusion

In Example 2, a check was made to see whether the novel fluorescent substrates FITC-ε-Acp-QG, Flc-QG, TR-β-Ala-QG and Dns-ε-Acp-QG would serve as substrates of MTG. A study was also made to see whether NK6-AP known as a good substrate of MTG and these fluorescent substrates would be crosslinked by MTG catalysis. The results obtained confirmed that all fluorescent substrates except Flc-QG allowed the reaction to proceed, indicating their ability to serve as substrates of MTG. The modified NK6-AP was subjected to activity measurement; the modification in no way deactivated the enzyme and it was site-specific for the tag. Since Flc-QG was not recognized as a substrate, the utility of a linker on the N terminal sided of Gln was suggested.

To show the independency of the fluorescent substrate on the origin of TGase that would recognize it, a study was also made to evaluate the reactivity of the fluorescent substrate for GTG, a TGase derived from the guinea pig liver. In the experiment, an evaluation was made to see whether FITC-β-Ala-QG would prove reactive in the TGase-catalyzed crosslinking reaction in the presence of β-casein known as a good substrate of TGase. FITC-derived fluorescence was observed in the lanes to which FITC-β-Ala-QG had been added, confirming that FITC-β-Ala-QG is also recognized by MTG; it might be possible to enhance the reactivity for GTG by changing the amino acid sequence on the C terminal side of Q.

### Example 3

### MTG-Catalyzed Introduction of Fluorescent Substrate into Antibodies

1. Introduction

In this Example, an attempt was made to introduce a fluorescent substrate directly into antibodies by MTG catalysis; the antibodies were yet to be subjected to recombination or fragmentation.

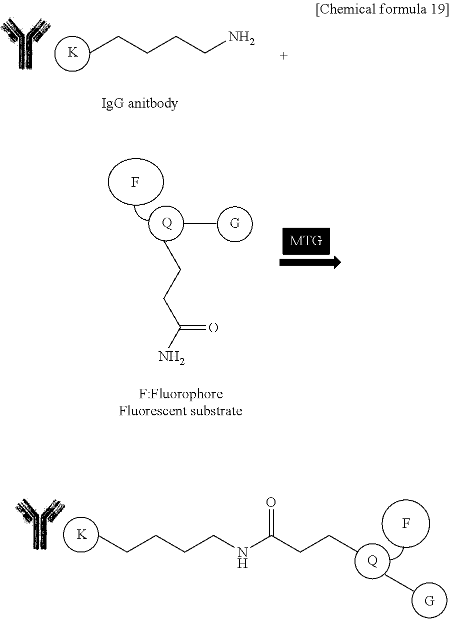

2. Experiment

2-1. Reagents

Various antibodies were purchased from COSMO BIO; the AP substrate ECF was purchased from GE Healthcare Bio-Science; 96-well microplates were purchased from Nunc; 1 ml HiTrap NHS-activated HP was purchased from GE Healthcare Bio-Science; ethanolamine was purchased from Wako. The other reagents were also commercially available.

2-2. MTG-Catalyzed Introduction of Fluorescent Substrates into Antibodies

A fluorescent substrate (FITC-ε-Acp-QG, FITC-β-Ala-QG, TR-β-Ala-QG or Dns-δ-Acp-QG), mouse-derived anti-lysozyme IgG2a antibody (monoclonal) and MTG were added to a 10 mM phosphate buffer (pH 7) to give respective final concentrations of 1 mM, 0.5 mg/ml and 1 U/ml. For the purpose of comparison, an MTG-free sample was prepared. The ingredients were thoroughly agitated and the resulting solution was left to stand overnight at 4° C.

After the reaction, the samples were subjected to SDS-PAGE. The gel from SDS-PAGE was examined with a fluorescent imager before it was stained with a CBB dye solution. For the Dns-ε-Acp-QG containing sample, examination was made with a transilluminator.

2-3. Evaluating Antigen Recognizing Ability by ELISA

A study was made on samples that were prepared by introducing a fluorescent substrate (FITC-ε-Acp-QG or FITC-β-Ala-QG) into a rabbit-derived anti-BSA IgG antibody (polyclonal) under the same conditions as in Experiment 2-2.

ELISA was performed by the following procedure (FIG. 3-5).
1) To a Black Plate, 5 mg/ml of BSA (antigen) was added in an amount of 150 l/well and left to stand overnight at 37° C.
2) The product of 1) was washed with PBST five times.
3) Modified anti-BSA IgG antibodies were added in an amount of 60 l/well and left to stand at room temperature for 5 hours.
4) The product of 3) was washed with PBST five times.
5) Examination was made with a fluorescent imager.
6) A 1.3 g/ml sample of AP-labeled anti-rabbit IgG IgG antibodies were added in an amount of 150 l/well and left to stand at 37° C. for 3 hours.
7) The product of 6) was washed with TBST five times.
8) The substrate was added and 10 minutes later, fluorescence derived from the enzymatic reaction was examined with a fluorescent imager.

2-4. Purification of Samples

The following buffers were preliminarily provided.
Coupling buffer (0.2 M NaHCO3, 0.5 M NaCl, pH 8.3)
Buffer A (0.5 M ethanolamine, 0.5 M NaCl, pH 8.3)
Buffer B (0.1 M acetate, 0.5 M NaCl, pH 4.0)
Elution buffer (0.1 M glycine-HCl, pH 3.0)

Described below is the procedure of immobilizing anti-mouse IgG IgG antibodies to HiTrap NHS-activated HP by chemical modification and purifying the product.
1) HiTrap NHS-activated HP was washed with 1 ml of cold 1 mM HCl six times.
2) Immediately after step 1), anti-mouse IgG IgG antibodies (10 mg) dissolved in the coupling buffer (1 ml) were added and reaction was performed at 25° C. for 2 hours.
3) To render an excess of active groups inactive and remove the uncoupled antibodies, washing was performed with buffer A, buffer B and buffer A in that order, six times each in an amount of 1 ml.
4) Reaction was performed at 25° C. for 2 hours.
5) Washing was performed with buffer B, buffer A and buffer B in that order, six times each in an amount of 1 ml.
6) PBS was added in an amount of 10 ml to equilibrate a column.
7) A sample having FITC-β-Ala-QG introduced into the antibodies by MTG catalysis was applied to the column.
8) PBS was added in an amount of 10 ml to wash the column.
9) The elution buffer was added in an amount of 3 ml to elute the antibodies.
10) After supplying the buffer A and buffer B alternately in an amount of 1 ml, PBS was added in an amount of 10 ml to achieve reequilibration.

3. Experimental Results and Discussion

3-1. Evaluating the Reactivity of Antibody with Fluorescent Substrates by MTG Catalysis

A Gln-containing fluorescent substrate and Lys in an antibody were reacted by MTG catalysis and after SDS-PAGE, fluorescence from the gel was observed for tracing purposes.

3-1-1. Reactivity of Antibody with FITC-ε-Acp-QG by MTG Catalysis

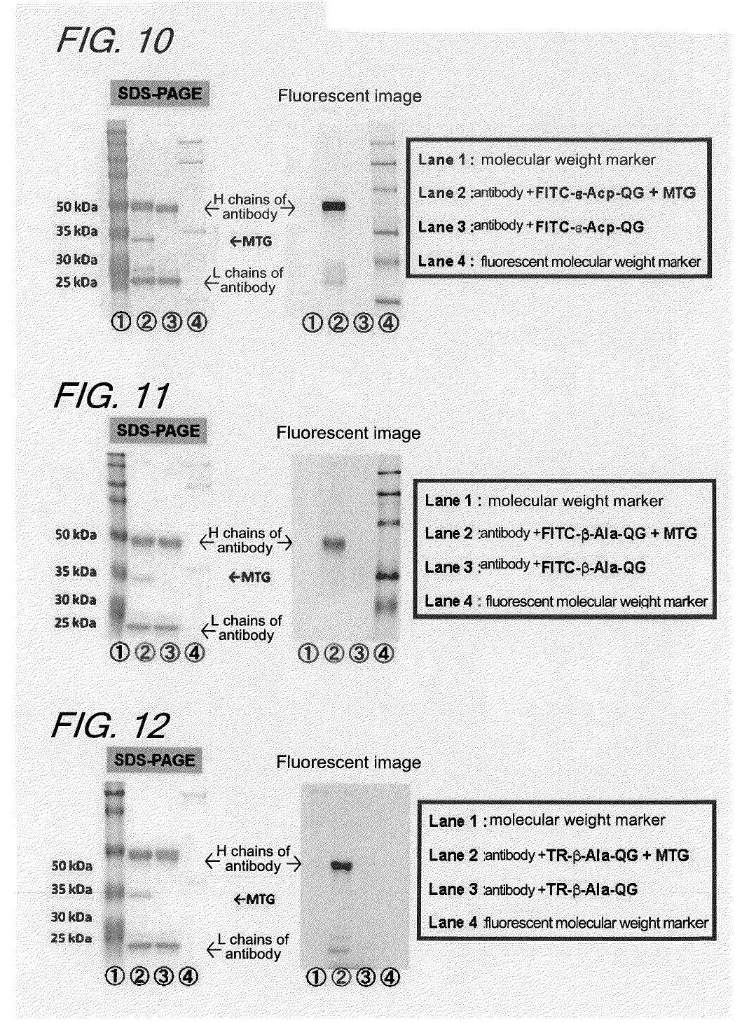

FIG. 10 shows the result of SDS-PAGE on samples after the reaction between FITC-ε-Acp-QG and an antibody, as well as the result of observing the gel from SDS-PAGE with a fluorescent imager. The IgG antibody is of such a structure that four polypeptide chains are coupled by disulfide bonds and has a molecular weight of about 150 kDa. The SDS-PAGE conducted in the experiment under consideration was of a reduced type, in which disulfide bonds were cleaved by mercaptoethanol. In SDS-PAGE, a band for the L chains was detected at ca. 25 kDa, a band for the H chains at ca. 50 kDa, and a band of MTG at ca. 38 kDa. The fluorescent images revealed that FITC-derived fluorescence was observed at ca. 50 kDa in the lane to which MTG had been added but no fluorescence was observed in the lane to which no MTG had been added. Thus, it is believed that the H chains of the antibody are modified with FITC-ε-Acp-QG by MTG catalysis.

3-1-2. Reactivity of Antibody with FITC-β-Ala-QG by MTG Catalysis

FIG. 11 shows the result of SDS-PAGE on samples after the reaction between FITC-β-Ala-QG and an antibody, as well as the result of observing the gel from SDS-PAGE with a fluorescent imager. In SDS-PAGE, a band for the L chains was detected at ca. 25 kDa, a band for the H chains at ca. 50 kDa, and a band of MTG at ca. 38 kDa. The fluorescent images revealed that FITC-derived fluorescence was observed at ca. 50 kDa in the lane to which MTG had been added but no fluorescence was observed in the lane to which no MTG had been added. Thus, it is believed that the H chains of the antibody are modified with FITC-β-Ala-QG by MTG catalysis.

3-1-3. Reactivity of TR-β-Ala-QG with Antibody by MTG Catalysis

FIG. 12 shows the result of SDS-PAGE on samples after the reaction between TR-β-Ala-QG and an antibody, as well as the result of observing the gel from SDS-PAGE with a fluorescent imager. In SDS-PAGE, a band for the L chains was detected at ca. 25 kDa, a band for the H chains at ca. 50 kDa, and a band of MTG at ca. 38 kDa. The fluorescent images revealed that fluorescence derived from Texas Red was observed at ca. 50 kDa in the lane to which MTG had been added but no fluorescence was observed in the lane to which no MTG had been added. Thus, it is believed that the H chains of the antibody are modified with TR-β-Ala-QG by MTG catalysis.

3-1-4. Reactivity of Antibody with Dns-ε-Acp-QG by MTG Catalysis

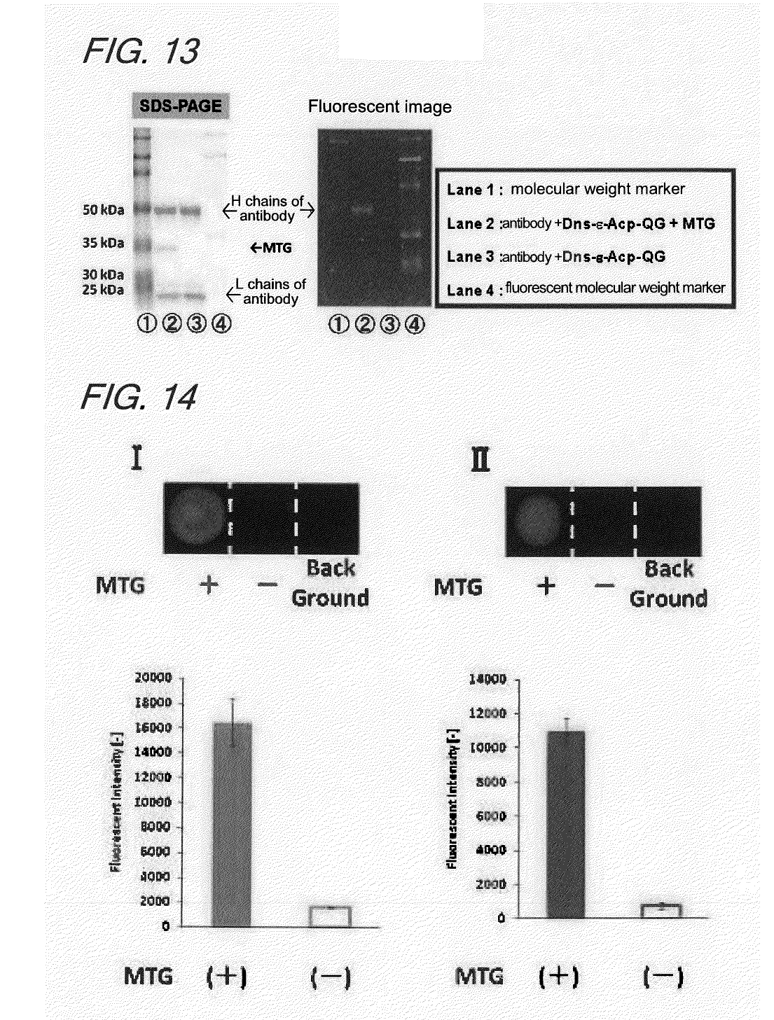

FIG. 13 shows the result of SDS-PAGE on samples after the reaction between Dns-ε-Acp-QG and an antibody, as well as the result of observing the gel from SDS-PAGE with a transilluminator. In SDS-PAGE, a band for the L chains was detected at ca. 25 kDa, a band for the H chains at ca. 50 kDa, and a band of MTG at ca. 38 kDa. The fluorescent images revealed that dansyl-derived fluorescence was observed at ca. 50 kDa in the lane to which MTG had been added but no fluorescence was observed in the lane to which no MTG had been added. Thus, it is believed that the H chains of the antibody are modified with Dns-ε-Acp-QG by MTG catalysis.

3-2. Verification of Antigen Recognizing Ability by ELISA

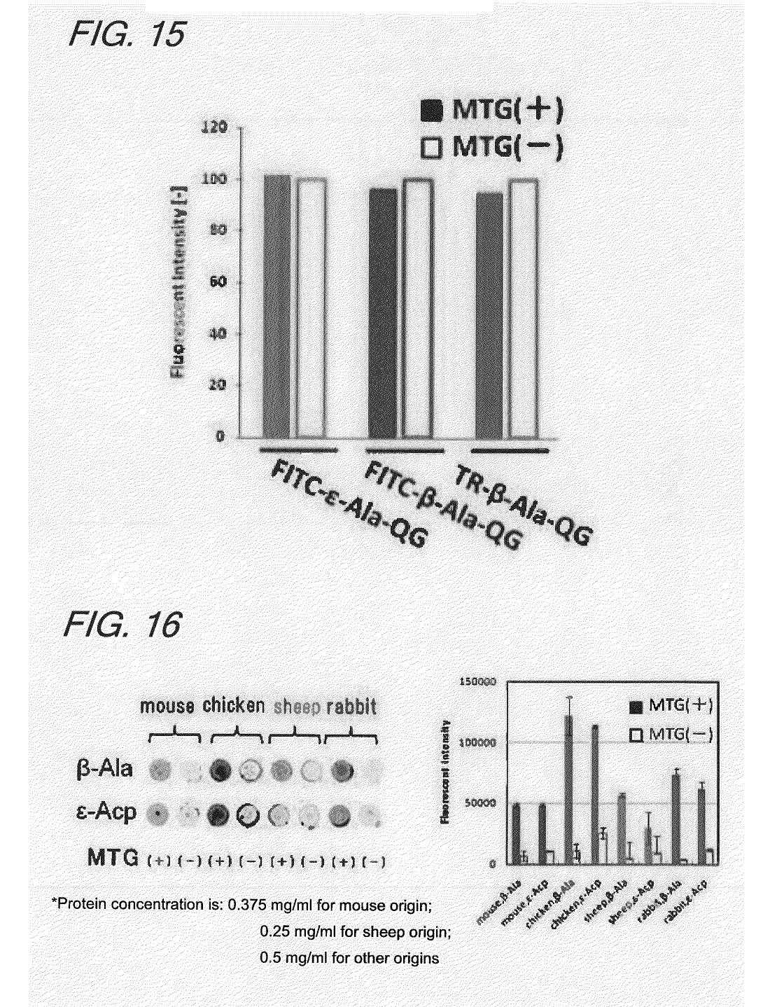

FIG. 14 shows the result of observation of fluorescence from a primary antibody with a fluorescent imager, as well as the result of fluorescent intensity measurement; FIG. 15 shows the result of the measurement with a fluorescent imager of the intensity of AP-derived fluorescence from a secondary antibody. On the basis of these results, an evaluation was made to see whether the antibodies into which the fluorescent substrates were introduced would maintain the antigen recognizing ability. As is clear from FIG. 14, fluorescence was observed in the MTG-loaded lanes for both FITC-ε-Acp-QG and FITC-β-Ala-QG and it was also clear from FIG. 15 that in the MTG-loaded wells, the primary antibody was detected in amounts that were comparable to those detected in the MTG-free well; it was thus verified that the modified antibody maintained its antigen recognizing ability.

4. Conclusion

In Example 3, a study was made to see whether fluorescent substrates could be introduced into an antibody by MTG catalysis. As it turned out, MTG catalysis was verified to enable site-specific modification of the H chains in the antibody with the fluorescent substrates tested (FITC-ε-Acp-QG, TR-β-Ala-QG, and Dns-ε-Acp-QG). The verification of antigen recognizing ability by ELISA showed no loss in the antigen recognizing ability due to the modification.

### Example 4

### Evaluating Reactivity for Antibodies Derived from Various Animal Species

1. Experiment

1-1. Reagents

Various antibodies were purchased from COSMO BIO. The other reagents were also commercially available.

1-2. MTG-Catalyzed Introduction of Fluorescent Substrates into Antibodies

A fluorescent substrate (FITC-β-Ala-QG or FITC-ε-Acp-QG), an anti-BSA IgG antibody (mouse-, sheep-, chicken- or rabbit-derived), and MTG were added to a 10 mM phosphate buffer (pH 7) to give final concentrations of 1 mM, 0.5 mg/ml, and 1 U/ml. For the purpose of comparison, an MTG-free sample was prepared. The ingredients were thoroughly agitated and the resulting solution was left to stand overnight at 4° C. for reaction.

1-3. Evaluating Antigen Recognizing Ability by ELISA

ELISA was performed by the following procedure.
1) To a Black Plate, 5 mg/ml of BSA (antigen) was added in an amount of 150 l/well and left to stand overnight at 37° C.
2) The product of 1) was washed with PBS five times.
3) Modified anti-BSA IgG antibodies were added in an amount of 60 l/well and left to stand at room temperature for 3 hours.
4) The product of 3) was washed with PBS five times.
5) Examination was made with a fluorescent imager.

2. Experimental Results and Discussion

2.1 Evaluating Reactivity for Antibodies Labeled by MTG Catalysis

FIG. 16 shows the result of ELISA observation with a fluorescent imager and the result of measurement of fluorescent intensity. On the basis of these results, an evaluation was made to determine whether the fluorescent substrates could be successfully introduced into the antibodies and whether the modified antibodies would maintain the antigen recognizing ability.

As it turned out, any of the MTG-loaded wells produced more intense fluorescence than the MTG-free wells. It was thus verified that the fluorescent substrates had been introduced into the antibodies by MTG catalysis and that the labeled antibodies maintained the antigen recognizing ability.

### Example 5

### Development of Novel Dual Labeled Substrate Capable of Recognition by MTG

1. Introduction

An attempt was made to develop a dual labeled substrate having a linker and a fluorescent group introduced on the N-terminal side of Gln and an aminated biotin introduced at the C-terminal of Gly. The fluorescent group has high detection sensitivity, provides a wide dynamic range of quantification, and also has a potential for imaging applications. Since biotin has an extremely high affinity for avidin (Ka=10¹⁵ M), the background can be reduced to provide improved detection sensitivity. Since these markers are introduced into a single substrate, the profile of activity vs immobilization yield can be plotted to calculate the specific activity of an enzyme; this is expected to contribute to the development of protein arrays by encouraging the quantitative discussion that has been a bottleneck in the art.

In the study under consideration, Dns-β-Ala-QG-biotin was designed and synthesized as a novel dual labeled substrate. Chosen as a model protein which was to be introduced was an alkaline phosphatase (NK6-AP) that is known to become a good substrate of MTG if a lysine tag is introduced at the N-terminal by a gene engineering technique and its reactivity was studied to evaluate the activity of AP that might be affected by the introduction. An attempt was also made to immobilize the dual labeled protein on an avidin-coated microplate. The following diagram shows in concept the study made in Example 5.

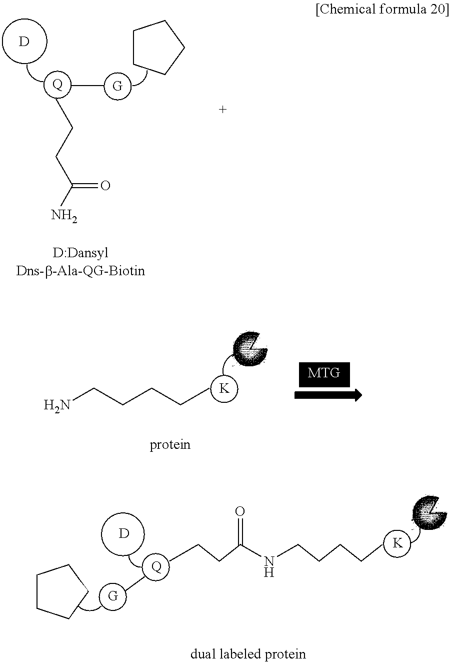

2. Experiment

2.1. Synthesis of Dns-β-Ala-QG-biotin

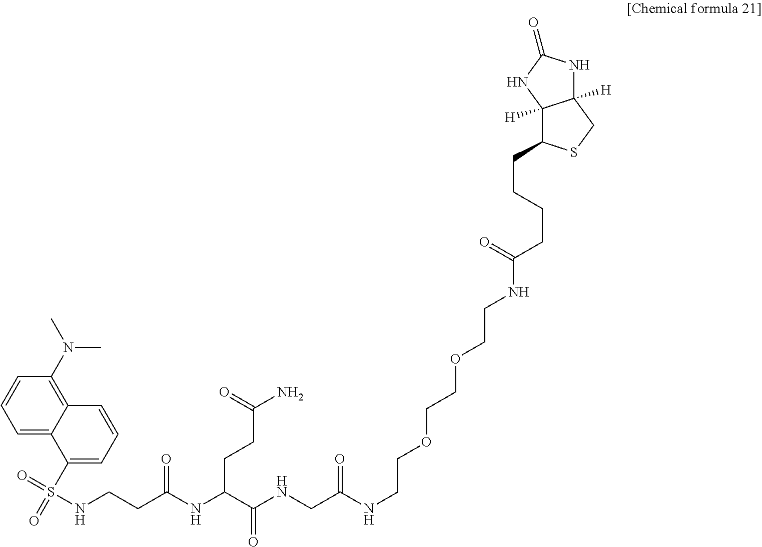

Dns-β-Ala-QG was synthesized by the procedure for the method of Fmoc solid-phase peptide synthesis described below. The amino acid Barlos Resin was used. Synthesis scale was 0.3 mmol

Procedure of peptide synthesis:
1) Assemble a PD-10 column and add a 0.3-mmol portion of amino acid Barlos Resin into it.
2) Wash with 5 ml of dichloromethane (DCM) three times.
3) Wash with 5 ml of dimethylformamide (DMF) three times.
4) Add 0.9 mmol of Fmoc-amino acid and 2.1 ml of a solution having 0.45 M HBTU/HOBt dissolved in DMF, as well as 2.1 ml of a solution having 0.9 M DIEA dissolved in DMF, and stir with a Vortex for a suitable period of time.
5) Wash with 5 ml of DMF five times. In this step, perform a Kaiser test to check for the presence of any unreacted resin. If the result is positive, return to step 4) and reextend the amino acid. If the result is negative, proceed to step 6).
6) Wash with 5 ml of DCM three times.
7) Wash with 5 ml of DMF three times.
8) Add 5 ml of a solution having 20% piperidine (PPD) dissolved in DMF, and stir for 1 minute to deprotect Fmoc.
9) Add 5 ml of a solution having 20% piperidine (PPD) dissolved in DMF, and stir for 15 minutes to deprotect Fmoc.
10) Wash with 5 ml of DMF three times.
11) Repeat steps 1) to 4) or steps 1) to 10) to extend the amino acid.
12) Add to the extended resin 0.9 mmol of dansyl-SO₃Cl and 2.1 ml of a solution having 0.45 M HBTU/HOBt dissolved in DMF, as well as 2.1 ml of a solution having 0.9 M DIEA dissolved in DMF and perform reaction for 1 hour.
13) Wash seven times with 5 ml of DMF, five times with 5 ml of DCM, and five times with 1 ml of methanol.
14) After washing, vacuum-dry overnight.
15) Add 1 ml of a cocktail consisting of TFA, TIS and deionized water at a ratio of 95:2.5:2.5 and stir for an hour to cut out slices from the resin.
16) Add diethyl ether to the solution of slices so that they precipitate; thereafter, repeat centrifugation and decantation cycles four times and vacuum-dry overnight.
17) Dissolve 10 mM Dns-β-Ala-QG, 10 mM aminated biotin, 10 mM HBTU, 10 mM HOBt and 20 mM DIEA in DMF and perform reaction.
18) Purify the resulting crud peptide by HPLC.
19) Verify the product by MALDI TOF-MS.

2-2. MTG-Catalyzed Introduction of Dns-β-Ala-QG-biotin into NK6-AP

Dns-β-Ala-QG-biotin, NK6-AP and MTG were added to 20 mM Tris-HCl (pH 8) to give respective final concentrations of 100 μM, 0.5 mg/ml, and 1 U/ml. For the purpose of comparison, an MTG-free sample was prepared. The ingredients were thoroughly agitated and the resulting solution was left to stand overnight at 4° C. for reaction.

2-3. Evaluating the Reactivity of MTG for Dns-β-Ala-QG-biotin

After the reaction, the samples were subjected to SDS-polyacrylamide gel electrophoresis (SDS-PAGE). The gel from SDS-PAGE was examined with a transilluminator before it was stained with Coomassie Brilliant Blue (CBB) in solution.

2-4. Evaluating the Effect of Modification on the Enzymatic Activity of NK6-AP

The activities of the samples prepared in Experiment 2-2 were measured, with p-nitrophenylphosphoric acid (p-NPP) being used as a substrate; AP would catalyze the dephosphorylation of p-NPP to form p-nitrophenol (p-NP). The reaction conditions for activity measurement were AP 28 nM, p-NPP 1 mM and pH 8.0 and at a controlled temperature of 27° C., the p-NP derived absorption at 410 nm was traced.

2-5. Purifying the Dual Labeled Protein by His-Tag Purification and Calculating the Degree of its Labeling

By His-tag purification on a Ni-NTA column, the dual labeled protein was separated from an excess of Dns-β-Ala-QG-biotin. An excess of the imidazole used in the elution step was removed by gel filtration purification on a PD-10 column with 20 mM Tris-HCl (pH 8) being used as an elution buffer.

The resulting dual labeled protein was concentrated with a suction-type centrifugal evaporator and an attempt was made to calculate the degree of its labeling by measuring the absorbance at 280 nm and 330 nm.

2-6. Immobilizing the Dual Labeled Protein on Microplate
1) After His-tag purification, the dual labeled protein (1.5 μM) was dissolved in 20 mM Tris-HCl (pH 8) and added onto an avidin-coated microplate (product of Nunc) in an amount of 100 μl/well so that it would be immobilized by the interaction between biotin and avidin (37° C., 1 h).
2) To observe the fluorescence derived from dansyl groups, 20 mM Tris-HCl (pH 8) was added in an amount of 100 μl/well and immobilization was verified by a fluorescent imager.
3) A solution of ECF as a fluorescent substrate of AP was diluted 100 folds with 1 M Tris-HCl (pH 8) and added onto the plate in an amount of 100 μl/well for reaction with the immobilized AP (25° C., 5 min).
   (Before steps 2) and 3), a microplate washer was used to wash the plate with TBST (TBS+5% Tween 20) three times each in an amount of 300 μl/well.)

3. Results and Discussion

3-1. Synthesis of Dns-β-Ala-QG-biotin

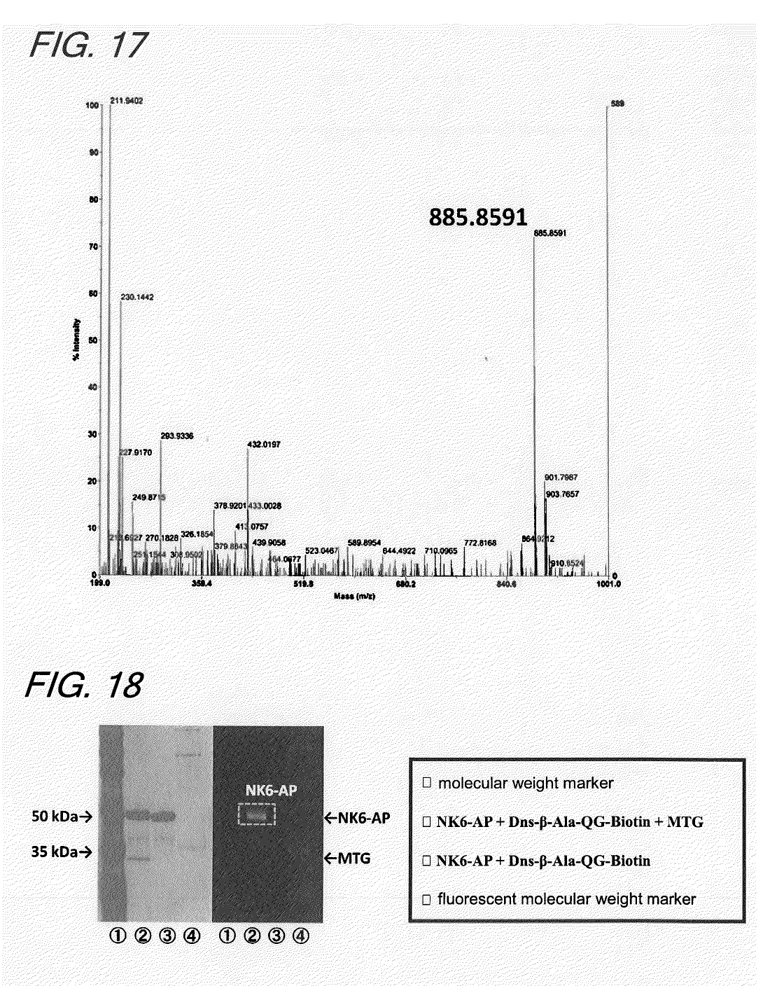

After purification by HPLC, MALDI TOF-MS was performed and a sharp peak was observed at 886 (FIG. 17). This agrees with the calculated value of the molecular weight of a sodium salt of Dns-β-Ala-QG-biotin (Mw: 886.36).

3-2. Evaluating the Reactivity of MTG for Dns-β-Ala-QG-biotin

Result of Observation by SDS-PAGE

FIG. 18 (left panel) shows the result of SDS-PAGE. A band for NK6-AP was observed at ca. 45 kDa and a band for MTG at ca. 35 kDa. In MTG-loaded lane (2), the molecular weight shifted somewhat toward the higher end, suggesting the introduction of Dns-β-Ala-QG-biotin into NK6-AP.

Result of Observation with a Fluorescent Imager

FIG. 18 (right panel) shows the result of examining the gel from SDS-PAGE with a transilluminator. Fluorescence derived from dansyl groups was observed in MTG-loaded lane (2) at ca. 45 kDa but no fluorescene was observed in MTG-free lane (3). It was therefore verified that Dns-β-Ala-QG-biotin had been introduced into NK6-AP.

3-3. Evaluating the Activity of Dual Labeled Protein that Might be Affected by MTG

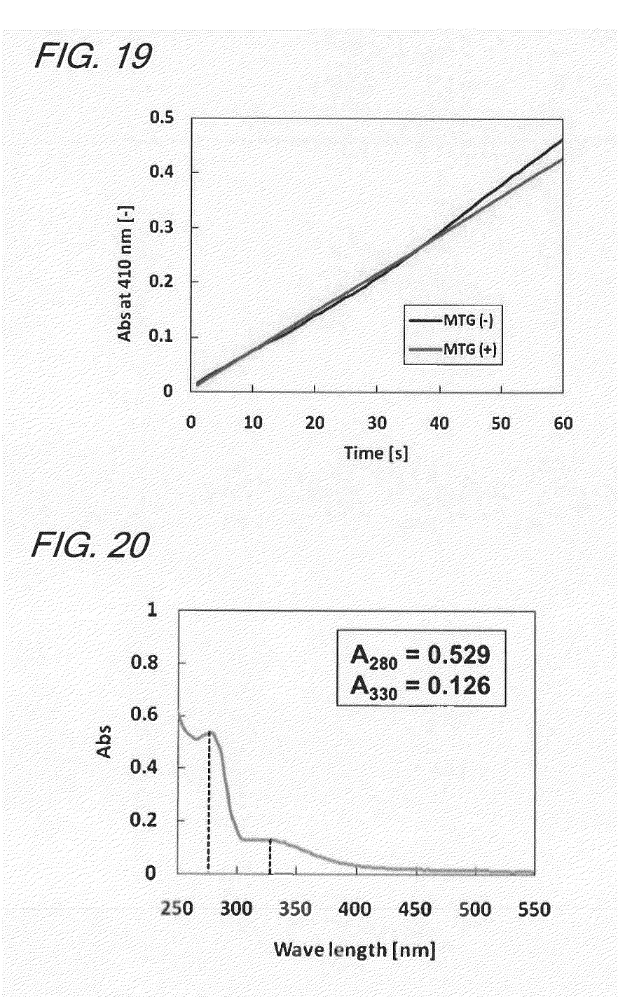

To study the effect that MTG-catalyzed introduction of Dns-β-Ala-QG-biotin might exert on a protein, the activity of NK6-AP was evaluated both before and after the introduction. The result is shown in FIG. 19. Obviously, there was hardly any difference in the activity of AP between the MTG-treated sample and the MTG-free sample. It was therefore suggested that Dns-β-Ala-QG-biotin could be introduced into NK6-AP without causing a functional loss from the modification.

3-4. Calculating the Degree of Labeling in the Dual Labeled Protein

After His-tag purification, the dual labeled protein was concentrated and the absorbance was measured at 280 nm and 330 nm to calculate the degree of labeling. FIG. 20 shows the result of measurement of the absorbance of the dual labeled protein. Assuming NK6-AP to have a molecular weight of 50,000 Da, NK6-AP and dansyl groups have the following molar extinction coefficients under the experimental conditions described above: ε₂₈₀=20000 [M⁻¹ cm⁻¹] and ε₃₃₀=550 [M⁻¹ cm⁻¹] for NK6-AP; ε₂₈₀=2460 [M⁻¹ cm⁻¹] and ε₃₃₀=4450 [M⁻¹ cm⁻¹] for dansyl groups. From these results, the degree of labeling is calculated as 1.1, indicating that Dns-β-Ala-QG-biotin was introduced into NK6-AP in a quantitative and highly efficient manner.

Degree of labeling=(Number of moles of dansyl groups)/(Number of moles of NK6-AP)=1.1≈1 [Formula 1]

3-5 Immobilizing the Dual Labeled Protein on Microplate

The dual labeled protein after His-tag purification was immobilized on an avidin-coated microplate. FIG. 21 (left panel) shows the result of measurement of fluorescence derived from the labeled dansyl groups and FIG. 21 (right panel) shows the result of measurement of relative activity derived from the immobilized NK6-AP. In both the right and left panels of FIG. 21, a significant difference in fluorescent intensity was observed in the dual labeled protein to which MTG had been added. The difference in fluorescent intensity observed in the left panel of FIG. 21 can be improved by changing the site of the fluorescent group. It is clear from the right panel of FIG. 21 that the signal-to-noise (SIN) ratio can be greatly improved by enzymatic signal amplification. From these findings, it can safely be concluded that the labeled dansyl groups emitted fluorescence and the protein was immobilized on the microplate while retaining its function through the interaction between biotin and avidin.

4. Conclusion

The present inventors successfully synthesized Dns-β-Ala-QG-biotin. They also confirmed the MTG-catalyzed introduction of Dns-β-Ala-QG-biotin into NK6-AP. The activity of NK6-AP was in no way affected by the introduction. The present inventors also successfully immobilized the dual labeled protein on the plate.

### Example 6

### Synthesis and Utilization of FITC-β-Ala-QG-Alkyne

1. Introduction

In this Example, a multifunctional peptide named “FITC-β-Ala-QG-alkyne” was synthesized with a view to achieving heteroconjugation; FITC-β-Ala-QG-alkyne was of such a structure that a linker and the fluorescent group FITC were introduced on the N-terminal side of Gln and an alkyne introduced at the C-terminal of Gly via PEG (see below).

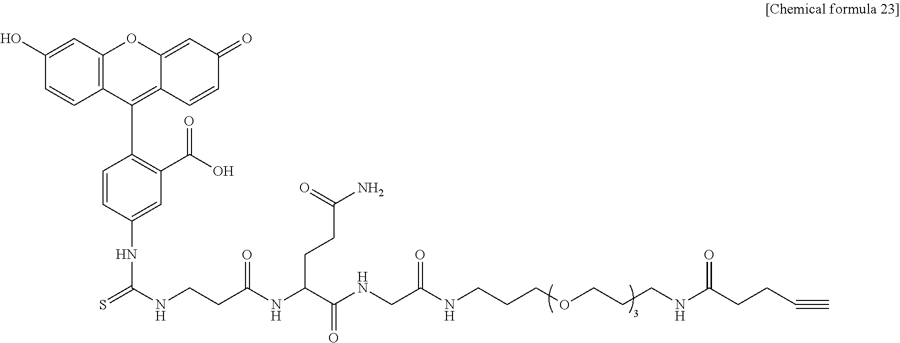

In this Example, the conditions for introducing FITC-β-Ala-QG into a K-tagged protein by MTG catalysis were optimized and, thereafter, an evaluation was made to see whether the synthesized FITC-β-Ala-QG-alkyne would be recognized by MTG. In another experiment, NK6-AP and BSA were used as model proteins and an attempt was made to see whether the azido derivative of BSA and NK6-AP into which FITC-β-Ala-QG-alkyne had been introduced could be conjugated by a click reaction. The following diagram shows in concept the study conduced.

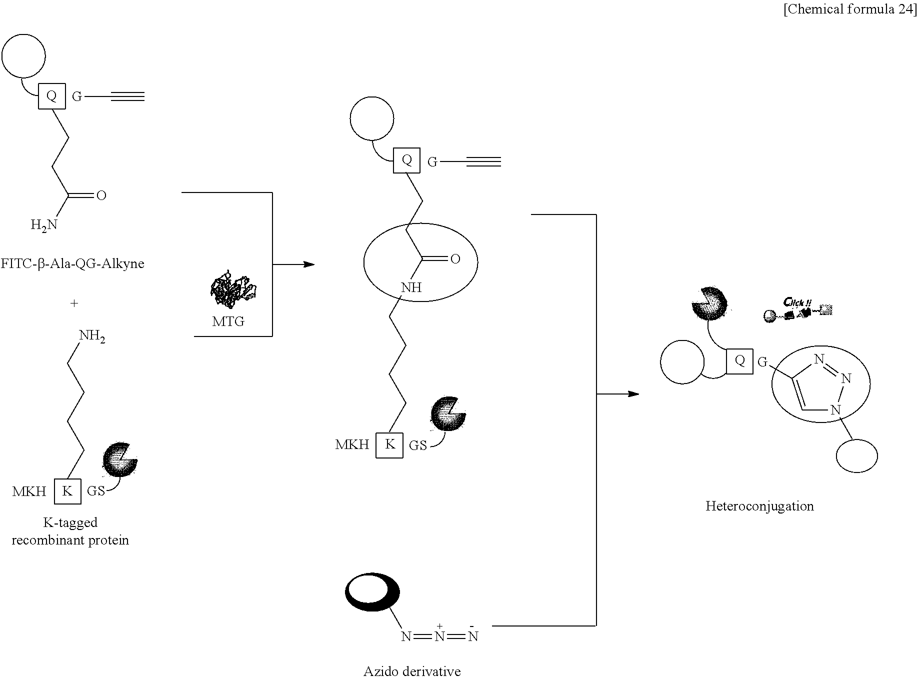

2. Experiment

2-1. Synthesis of FITC-β-Ala-QG-alkyne

FITC-β-Ala-QG-alkyne was synthesized in accordance with the procedure of Fmoc solid-phase synthesis described below. Universal-PEG NovaTag^{TR} Resin was used (see below). Synthesis scale was 0.083 mmol

25 Procedure of Peptide Synthesis
1) Assemble a PD-10 column and add a 0.083-mmol portion of Universal-PEG NovaTag^{TR} Resin into it.
2) Swell the resin with 5 ml of dimethylformamide (DMF) for 30 minutes.
3) Wash with 3 ml of dimethylformamide (DMF) three times.
4) Stir in 1M HOBt (dichloromethane (DCM)/TFE=1:1) for an hour to deprotect S-Mmt groups.
5) Wash with 3 ml of DCM three times.
6) Wash with 3 ml of DMF three times.
7) Add 0.3 mmol 4-pentynoic acid, 0.3 mmol HBTU and 0 6 mmol HOBt and perform reaction for an 1 hour.
8) Add 5 ml of a solution of 20% piperidine (PPD) in DMF and stir for a minute to deprotect Fmoc.
9) Add 5 ml of a solution of 20% piperidine (PPD) in DMF and stir for 15 minutes to deprotect Fmoc.
10) Wash with 3 ml of DMF five times.

Subsequently, Gly, Gln, β-Ala and FITC were extended in that order and the product was cut out and purified by HPLC. Its quality was verified by MALDI TOF-MS.

2-2. Optimization of pH Conditions in MTG-Catalyzed Introduction of FITC-β-Ala-QG into NK6-AP

To a 50 mM buffer whose pH was varied over the range of from 5 to 9, FITC-β-Ala-QG, NK6-AP and MTG were added to give respective final concentrations of 0.1 mM, 0.5 mg/ml, and 0.1 U/ml. These ingredients were well stirred and left to stand at 4° C. for reaction. After 6-hr or 15-minute reaction, the sample was mixed with a sample buffer and heated at 95° C. for 15 minutes to stop the reaction. Thereafter, SDS-polyacrylamide gel electrophoresis (SDS-PAGE) was applied. The gel from SDS-PAGE was stained with Coomassie Brilliant Blue (CBB) and observed with a fluorescent imager.

2-3. Optimization of MTG Concentration in MTG-Catalyzed Introduction of FITC-β-Ala-QG into NK6-AP

To 50 mM Tris-HCl (pH 8), FITC-β-Ala-QG, NK6-AP and MTG were added to give respective final concentrations of 0.1 mM, 0.5 mg/ml, and 0.001-1 U/ml. These ingredients were well stirred and left to stand at 4° C. for reaction. The reaction was stopped and evaluation was made by the same procedures as in Experiment 2-2.

2-4. MTG-Catalyzed Introduction of FITC-β-Ala-QG-alkyne into NK6-AP

To 50 mM Tris-HCl (pH 8), FITC-β-Ala-QG-alkyne, NK6-AP and MTG were added to give respective final concentrations of 0.1 mM, 0.5 mg/ml, and 0.1 U/ml. For the purpose of comparison, a MTG-free sample was also prepared. Both samples were well stirred and left to stand at 4° C. for reaction. The reaction was stopped and evaluation was made by the same procedures as in Experiment 2-2.

2-5. Evaluating the Effect of Modification on the Enzymatic Activity of NK6-AP

With p-nitrophenylphosphoric acid (p-NPP) being used as a substrate, the sampled prepared in Experiment 2-4 were measured; AP would catalyze the dephosphorylation of p-NPP to form p-nitrophenol (p-NP) (see below). The reaction conditions for activity measurement were AP 28 nM, p-NPP 1 mM and pH 8.0 and at a controlled temperature of 27° C., the p-NP derived absorption at 410 nm was traced.

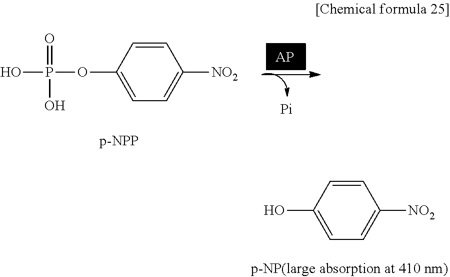

2-6. Purifying the Alkyne-Modified Protein by His-Tag Purification and Calculating the Degree of its Labeling

By His-tag purification on a Ni-NTA column, the alkyne-modified protein was separated from an excess of FITC-β-Ala-QG-alkyne. An excess of the imidazole used in the elution step was removed by gel filtration purification on a PD-10 column with 20 mM Tris-HCl (pH 8) being used as an elution buffer.

The resulting alkyne-modified protein was concentrated by ultrafiltration and the degree of its labeling was calculated by measuring the absorbance at 280 nm (derived from NK6-AP) and 330 nm (from FITC).

2-7. Introduction of NHS-Treated Azide into BSA by Chemical Modification

To a 200 mM carbonate buffer (pH 8.3), BSA and NHS-treated azide were added to give respective final concentrations of 0.5 mg/ml and 1 mM. These ingredients were well stirred and left to stand at 4° C. for 12 hours to perform reaction.

2-8. Conjugation Between Proteins by Click Reaction

An attempt was made to see whether the alkyne-modified AP prepared in Experiment 2-6 and the azide derivative of BSA prepared in Experiment 2-7 could be conjugated by a click reaction. The reaction conditions are set forth in the following table. Two comparative samples were also prepared; one was AP into which FITC-β-Ala-QG-alkyne was not introduced, and the other was a CuSO₄-free sample.

TABLE 2
Conditions of click reaction

| | |
|---|---|
| Alkyne-modified AP | 0.1 mg/ml |
| Azide derivative of BSA | 0.1 mg/ml |
| CuSO₄ | 2 mM |
| TCEP | 4 mM |
| TBTA | 0.2 mM |
| 50 mM Tris-HCl (pH 8) | |

3. Results and Discussion

3-1. Synthesis of FITC-β-Ala-QG-alkyne

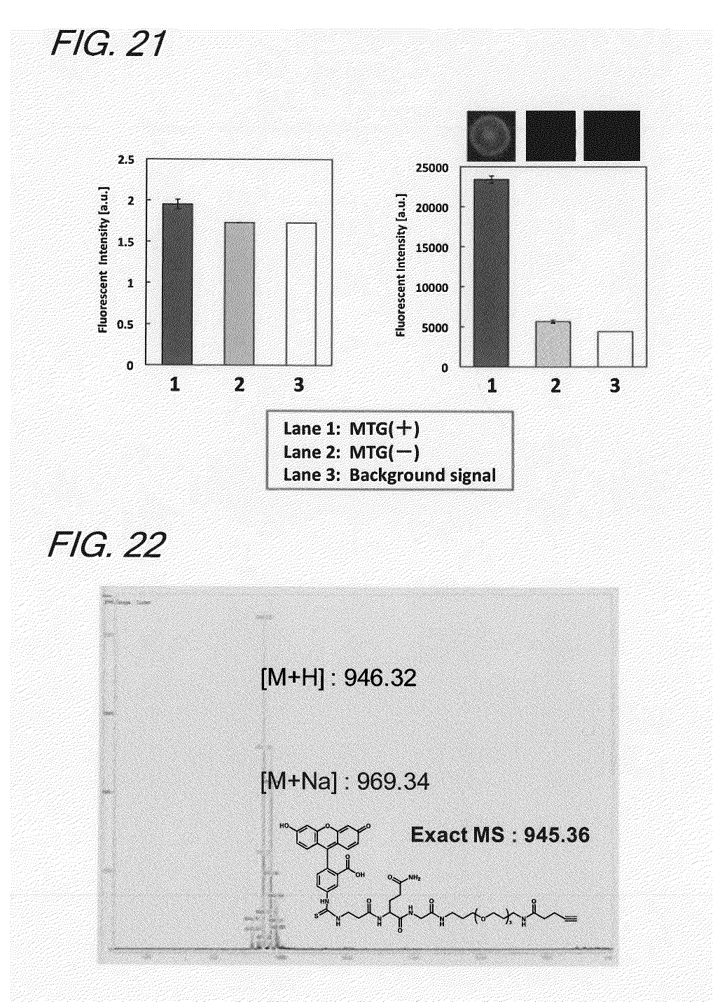

FIG. 22 shows the structure of FITC-β-Ala-QG-alkyne and the result of MALDI TOF-MS performed after its purification by HPLC. Obviously, two sharp peaks were observed at 946.32 for [M+H] and 969.34 for [M+Na]. This agreed with the molecular weight of FITC-β-Ala-QG-alkyne (Exact MS: 945.36).

3-2. Optimization of pH Conditions in MTG-Catalyzed Introduction of FITC-β-Ala-QG into NK6-AP

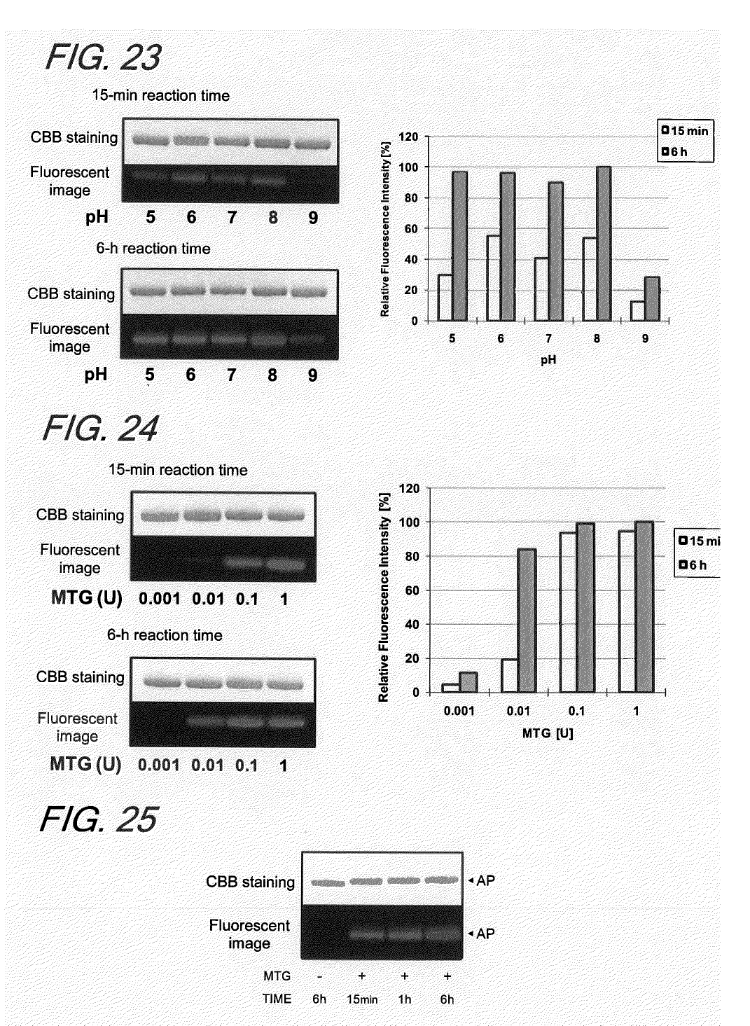

FIG. 23 shows the results of the study conducted to know optimum pH conditions by SDS-PAGE performed on the samples subjected to reaction for 15 minutes and 6 hours, respectively, and a fluorescent intensity analysis on the gel from SDS-PAGE. In the 15-min sample, rapid introduction was achieved at pH values of 6 and 8. This would be due to the inclusion of two factors in the MTG-catalyzed introduction of FITC-β-Ala-QG into NK6-AP. As the first factor, the protonation of the fluorescein in FITC-β-Ala-QG (FITC: pKa 6.4) at lower pH might have rendered it more hydrophobic. The substrate specificity of MTG is known to be such that it prefers the N-terminal side of a Gln residue to be in a bulky and hydrophobic environment (see Non-Patent Document 4, supra) and a drop in the Km value might have improved the efficiency of introduction. As the second factor, the deprotonation of the fluorescein in FITC-β-Ala-QG near pH 8 might have rendered it more anionic. The substrate specificity of MTG is also known to be such that the Lys residue (or primary amine) enters a negatively charged cleft in MTG to make a nucleophilic attack on the acyl intermediate, with the result that the substrate becomes more reactive on account of the presence of a positively charged amino acid residue (S. Taguchi, K. I. Nishihama, K. Igi, K. Ito, H. Taira, M. Motoki, H. Momose; J. Biochem., 2000, 128, 415-425). In this case, it is also suggested that as the result of the increase in anionic nature due to the deprotonation of fluorescein, FITC-β-Ala-QG might have interacted with the amino acid bearing the positive charge of K-tag, thus contributing to an improvement in reactivity. At pH 9, the cationic nature of K-tag might have weakened and the electrostatic proximity effect might have also weakened since MTG has a pI of 8.9 (Ap has a pI of 4.5). In the 6-hr sample, comparable levels of fluorescent intensity were observed in the pH range of 5-8, suggesting the reaction was saturated.

3-3. Optimization of MTG Concentration in MTG-Catalyzed Introduction of FITC-β-Ala-QG Into NK6-AP

FIG. 24 shows the results of the study conducted to know optimum MTG concentrations by SDS-PAGE performed on the samples subjected to reaction for 15 minutes and 6 hours, respectively, and a fluorescent intensity analysis on the gel from SDS-PAGE. In MTG catalysis, the water molecule also works as an acyl acceptor to cause a competitive reaction in which Gln is hydrolyzed and deamidated to Glu. Hence, an excessively high MTG concentration potentially increases the chance of a side reaction to occur and it is necessary to determine an appropriate MTG concentration. As it turned out, an adequately rapid reaction proceeded at 0.1 U. The degree of introduction did not drop at 1 U. This is probably because the reactivity between FITC-β-Ala-QG and K-tag was so high that their reaction proceeded more favorably than the reaction with the water molecule.

3-4. Evaluating the Reactivity of MTG for FITC-β-Ala-QG-alkyne

FIG. 25 shows the results of SDS-PAGE performed on samples after 15-min, 1-hr, and 6-hr reactions. No FITC-derived fluorescence was found in the AP band in the MTG-free lane whereas fluorescence was found in the AP bands in the MTG-loaded lanes. It was therefore verified that FITC-β-Ala-QG-alkyne had been introduced into NK6-AP by MTG catalysis. The reaction time was also comparable to the case of FITC-β-Ala-QG, confirming the rapidity of introduction.

3-5. Evaluating the Activity of Alkyne-Modified Protein that Might be Affected by MTG

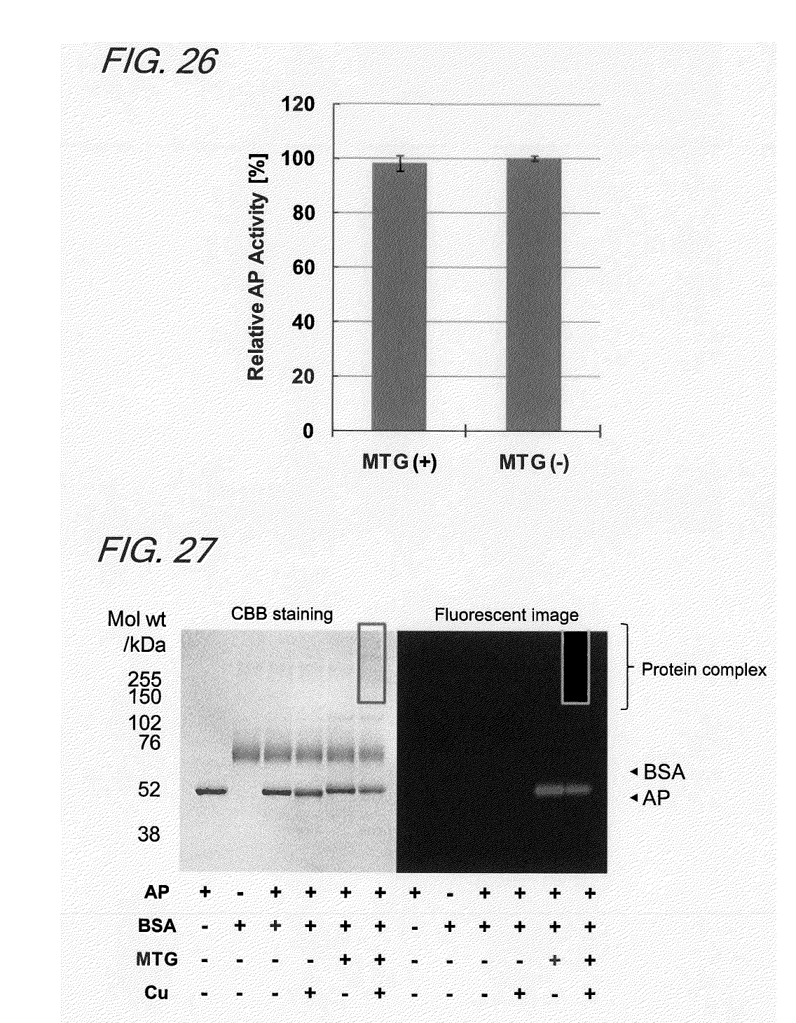

To study the effect that MTG-catalyzed introduction of FITC-β-Ala-QG-alkyne might exert on a protein, the activity of NK6-AP was evaluated both before and after the introduction. As shown in FIG. 26, the MTG-treated sample hardly experienced a change in the activity of AP, demonstrating that FITC-β-Ala-QG-alkyne could be introduced into NK6-AP without experiencing a functional loss.

3-6. Conjugation Between Proteins by Click Reaction

FIG. 27 shows the result of SDS-PAGE performed on the samples with which the present inventors attempted to conjugate the alkyne-modified AP and the azide derivative of BSA by a click reaction. No reaction for protein conjugation was found to occur in the sample using AP into which no FITC-β-Ala-QG-alkyne had been introduced and in the samples to which no CuSO₄ had been added; in contrast, an increase in the molecular weight due to the conjugation between proteins was found to occur only in the sample that used AP having FITC-β-Ala-QG-alkyne introduced thereinto by MTG catalysis and to which CuSO₄ had been added. This demonstrated that the alkyne introduced by MTG catalysis and the azide derivative of protein were conjugated by a click reaction.

4. Conclusion

The present inventors successfully synthesized FITC-β-Ala-QG-alkyne. They confirmed that FITC-β-Ala-QG-alkyne was introduced into AP while hardly affecting its activity. It was also confirmed that the protein labeled with FITC-β-Ala-QG-alkyne could be conjugated to the azide derivative of protein by a click reaction.

### Example 7

### Optimization of Antibody Labeling Conditions and Immobilization of Antibodies on Avidin-Coated Resin through Biotin Labeling

1. Introduction

In this Example, a multilabeled substrate FITC-β-Ala-QG-biotin having the fluorochrome FITC introduced on the N-terminal side of Gln and biotin introduced at the C-terminal of Gly (see below) was tested to determine optimum conditions for its introduction into an antibody and to thereby improve the degree of its labeling. Finally, with a view to developing a technique of manufacturing antibody arrays, an attempt was made to immobilize the antibody on an avidin-coated resin via biotin.

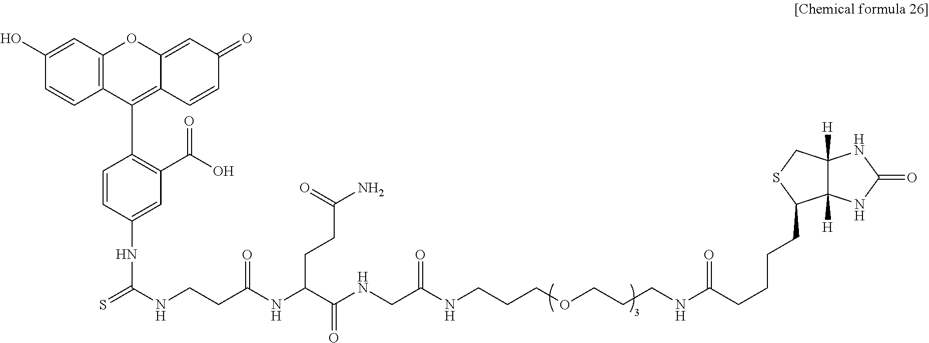

The concept of the present study is shown below.

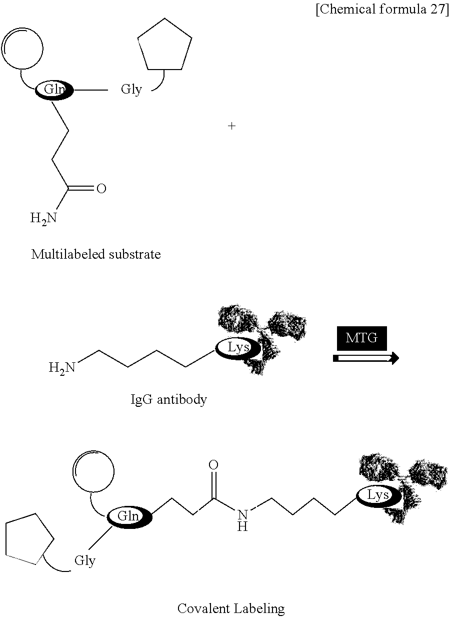

2. Experiment

2-1. Optimization of pH Conditions in MTG-Catalyzed Introduction of FITC-β-Ala-QG into Antibody

To a 50 mM buffer whose pH was varied over the range from 5 to 9 (pH 5 acetate buffer; pH 6-7 phosphate buffer; pH 8 Tris-HCl; pH 9 boric acid buffer), FITC-β-Ala-QG, an anti-PSA (prostate specific antigen) IgG antibody (mouse-derived, monoclonal) and MTG were added to give respective final concentrations of 0.1 mM, 0.5 mg/ml, and 1 U/ml. These ingredients were well stirred and left to stand at 4° C. for reaction. After 1-hr or 6-hr reaction, the sample was mixed with a sample buffer and heated at 95° C. for 15 minutes to stop the reaction. Thereafter, SDS-polyacrylamide gel electrophoresis (SDS-PAGE) was applied. The gel from SDS-PAGE was stained with Coomassie Brilliant Blue (CBB) and observed with a fluorescent imager.

2-2. Optimization of MTG Concentration in MTG-Catalyzed Introduction of FITC-β-Ala-QG into Antibody

To 50 mM Tris-HCl (pH 8), FITC-β-Ala-QG, an anti-PSA IgG antibody and MTG were added to give respective final concentrations of 1 mM, 0.5 mg/ml, and 0.001-1 U/ml. These ingredients were well stirred and left to stand at 4° C. for reaction. The reaction was stopped and evaluation was made by the same procedures as in Experiment 2-1.

2-3. Optimization of Substrate Concentration in MTG-Catalyzed Introduction of FITC-β-Ala-QG into Antibody

To 50 mM Tris-HCl (pH 8), FITC-β-Ala-QG, an anti-PSA IgG antibody and MTG were added to give respective final concentrations of 0.001-1 mM, 0.5 mg/ml, and 1 U/ml. These ingredients were well stirred and left to stand at 4° C. for reaction. The reaction was stopped and evaluation was made by the same procedures as in Experiment 2-1.

2-4. Optimization of Reaction Temperature in MTG-Catalyzed Introduction of FITC-β-Ala-QG into Antibody

To PBS, FITC-β-Ala-QG, an anti-PSA IgG antibody (mouse-derived, monoclonal) and MTG were added to give respective final concentrations of 1 mM, 0.5 mg/ml, and 1 U/ml. These ingredients were well stirred and left to stand at 4° C. or 40° C. for reaction. After 3-, 6- and 18-hr reactions, the reaction was stopped and evaluation was made by the same procedures as in Experiment 2-1.

2-5. MTG-Catalyzed Introduction of FITC-β-Ala-QG-biotin into Antibody

To PBS, FITC-β-Ala-QG, an anti-PSA IgG antibody (mouse-derived, monoclonal) and MTG were added to give respective final concentrations of 1 mM, 0.5 mg/ml, and 1 U/ml. These ingredients were well stirred and left to stand for 18 hours at 4° C. for reaction. For the purpose of comparison, two additional samples were prepared, one being free of FITC-β-Ala-QG-biotin and the other having been reacted with a biotin-free fluorescent substrate (FITC-β-Ala-QG). After the reaction, the samples were purified by ultrafiltration (product of MILLIPORE Corp.; 100 kDa cut).

2-6 Immobilization of Multilabeled Anti-BSA IgG Antibodies on Avidin-Coated Resin

The multilabeled antibodies were immobilized on an avidin-coated resin by the procedure described below (FIG. 4).
1) An avidin-coated resin (UltraLink Immobilized Streptavidin Plus; product of PIERCE) (100 μl) was washed with PBS (binding buffer) (1 ml) twice.
2) Each sample (0.25 mg/ml) weighing 100 μl was added to the resin and stirred for 30 minutes.
3) To recover the supernatant, the resin was allowed to settle by centrifugation and the supernatant was recovered.
4) Two washings were performed with PBS (1 ml).
5) To the resin, a 2× sample buffer (20 μl) was added and the mixture was heated up to 90° C. to elute the multilabeled antibodies from the resin.
6) The sample before the addition to the resin, the supernatant recovered in 3) and the multilabeled antibodies eluted in 4) were identified by SDS-PAGE.

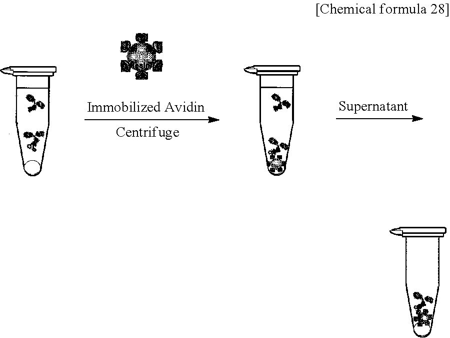

Procedure of immobilizing multilabeled anti-BSA IgG antibodies on avidin-coated resin

3. Results and discussion

3-1. Optimization of pH Conditions in MTG-Catalyzed Introduction of FITC-β-Ala-QG into Antibody (Anti-PSA IgG Antibody)

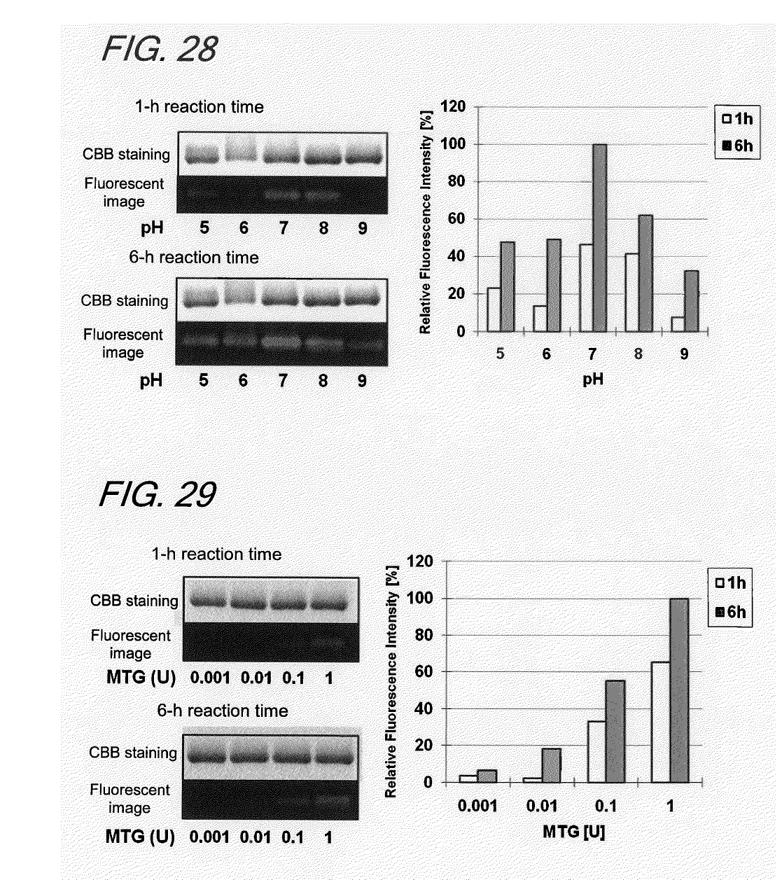

FIG. 28 shows the results of the study conducted to know optimum pH conditions by SDS-PAGE performed on the samples subjected to reaction for 1 hours and 6 hours, respectively, and a fluorescent intensity analysis on the gel from SDS-PAGE. The foregoing studies have shown that the reaction would proceed in a manner specific to the H chains of the antibody and the result of SDS-PAGE shows bands for the H chains of the antibody. Obviously, highly efficient introduction of FITC-β-Ala-QG was verified to occur near pH 7. This optimum pH for the reaction between the antibody and FITC-β-Ala-QG agreed with the optimum pH for MTG as determined by the hydroxamate method (H. Ando, M. Adachi, K. Umeda, A. Matsuura, M. Nonaka, R. Uchio, H. Tanaka, M. Motoki; Agric. Biol. Chem., 1989, 53, 2613-2617).

3-2. Optimization of MTG Concentration in MTG-Catalyzed Introduction of FITC-β-Ala-QG into Antibody (Anti-PSA IgG Antibody)

FIG. 29 shows the results of the study conducted to know optimum MTG concentrations by SDS-PAGE performed on the samples subjected to reaction for 1 hour and 6 hours, respectively, and a fluorescent intensity analysis on the gel from SDS-PAGE. In MTG catalysis, the water molecule also works as an acyl acceptor to cause a competitive reaction in which Gln is hydrolyzed and deamidated to Glu. Hence, an excessively high MTG concentration potentially increases the chance of a side reaction to occur and it is necessary to determine an appropriate MTG concentration. As it turned out, a highly efficient reaction proceeded at 1 U. Probably on account of the low reactivity of Lys in the antibody, highly efficient labeling would not proceed without an adequate level of MTG concentration.

3-3. Optimization of Substrate Concentration in MTG-Catalyzed Introduction of FITC-β-Ala-QG into Antibody (Anti-PSA IgG Antibody)

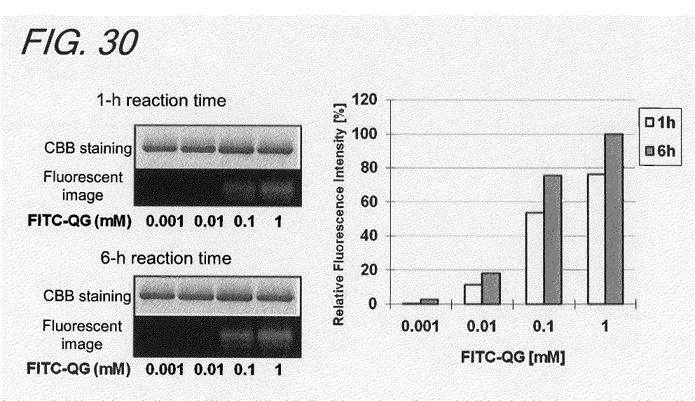

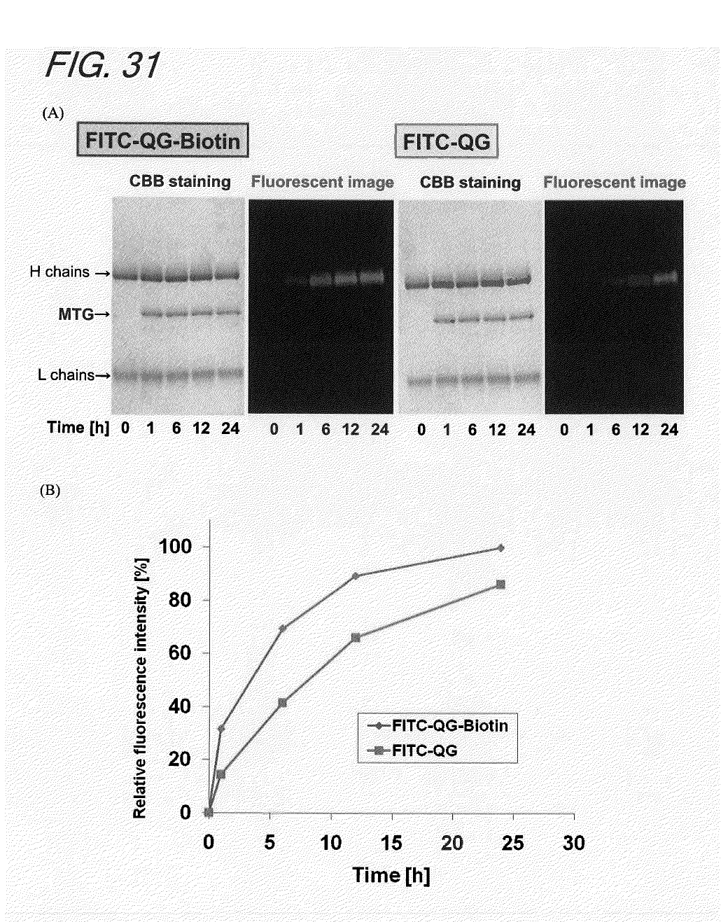

FIG. 30 shows the results of the study conducted to know optimum MTG concentrations by SDS-PAGE performed on the samples subjected to reaction for 1 hour and 6 hours, respectively, and a fluorescent intensity analysis on the gel from SDS-PAGE. As in the study for optimizing the MTG concentration, the labeling of the antibody proceeded with the increasing substrate concentration.

3-4. Comparative Study on the Labeling of Antibody with the Novel Fluorescent Substrates (Anti-PSA IgG Antibody)

In the studies described in 3-1 to 3-3, system optimization was performed. As the final study, comparison of reactivity was made between different substrates. FIG. 31 shows the results of SDS-PAGE performed on the samples subjected to reaction for 0, 1, 6, 12 and 24 hours, respectively, and a fluorescent intensity analysis on the gel from SDS-PAGE. Obviously, introduction of biotin into FITC-β-Ala-QG did not impair the reactivity for the labeling of antibody (FIG. 31(A)). The plotting of a time-dependent change in the 24-hr reaction on a fluorescent intensity basis revealed a quicker response of FITC-β-Ala-QG-biotin than FITC-β-Ala-QG (FIG. 31(B)). These findings clearly show that introducing a new functionality at the C terminal of the novel fluorescent substrates is not deleterious to MTG-catalyzed labeling.

3-5. Immobilizing the Multilabeled Antibody to Avidin-Coated Resin (Anti-BSA IgG Antibody)

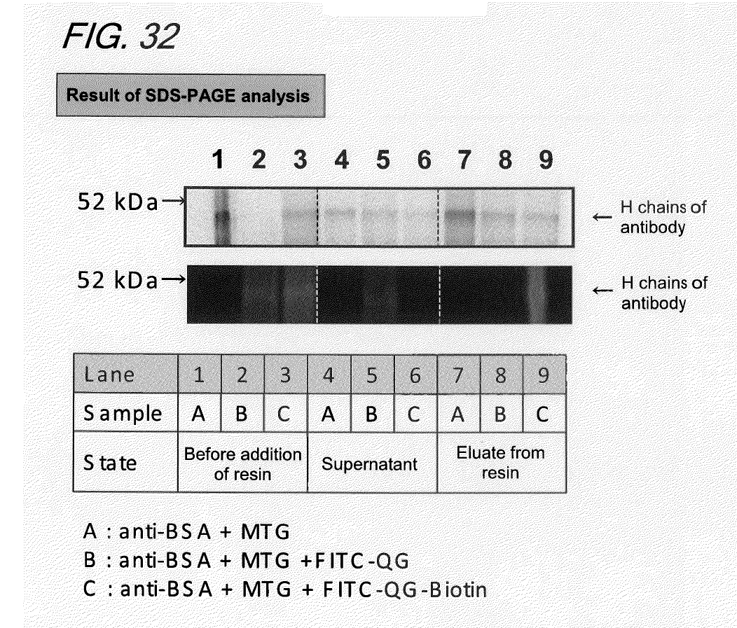

The present inventors had heretofore been unable to verify the coupling of the multilabeled antibody to avidin, namely, the introduction of a biotin group into the multilabeled antibody. In the present invention, an attempt was made to optimize the labeling conditions and immobilize the multilabeled antibody on the avidin-coated resin. The results are shown in FIG. 32. The data before the addition of the resin (lanes 1-3) verified the introduction of both FITC-β-Ala-QG and FITC-β-Ala-QG-biotin into the antibody. Subsequently, the avidin-coated resin was added to perform reaction and, thereafter, the resin was allowed to settle by centrifugation and the supernatant was analyzed to give the results shown as lanes 4-6. Lane 5 showing FITC-derived fluorescence suggests that the FITC-β-Ala-QG labeled antibodies were not bound to the avidin-coated resin but recovered in the supernatant whereas lane 6 showing no fluorescence suggests that the multilabeled antibodies bound to the avidin-coated resin. As a final step, the avidin-coated resin was dispersed in a sample 1 buffer and heated to liberate the multilabeled antibodies from avidin; analysis of the liberated antibodies gave the results shown as lanes 7-9. In lane 9, strong FITC-derived fluorescence was observed, probably due to the liberation of the multilabeled antibodies from the avidin-coated resin. It was therefore confirmed that the fluorochrome FITC and the ligand biotin group had been introduced into the antibodies.

4. Conclusion

The conditions for introducing FITC-β-Ala-QG into antibodies by MTG catalysis were optimized. Multilabeled antibodies could successfully be immobilized on the avidin-coated resin.

1. A substrate of TGase represented by (fluorescent group)-(linker)-(a portion containing a Gln residue capable of recognition by transglutaminase (TGase))-R (wherein:
the fluorescent group is fluorescein isothiocyanate (FITC), Texas Red (1E) or dansyl (Dns) or a group derived therefrom;
the linker is a group represented by —NH—(CH₂)ₙ—CO— (n is an integer of 1 to 6);
the portion containing a Gln residue capable of recognition by TGase is QX (X is an amino acid residue other than lysine (Lys)); and
R is a hydroxyl group, or biotin, nucleic acid, polyethylene glycol, azide, alkyne, maleimide or cyclopentadiene, or a group derived therefrom.)

2. The substrate of TGase according to claim 1 wherein the portion containing a Gln residue capable of recognition by TGase is QG and R is a hydroxyl group or a group derived from biotin or alkyne.

3. The substrate of TGase according to claim 2 which is one member of the group consisting of:

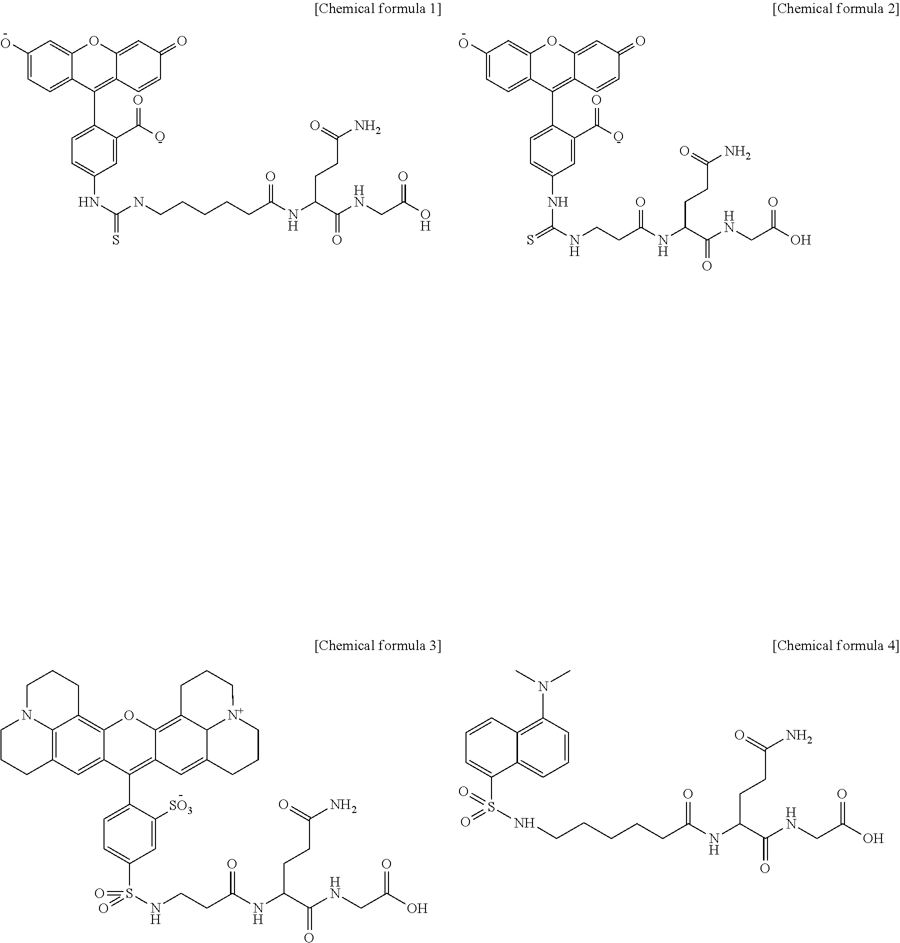

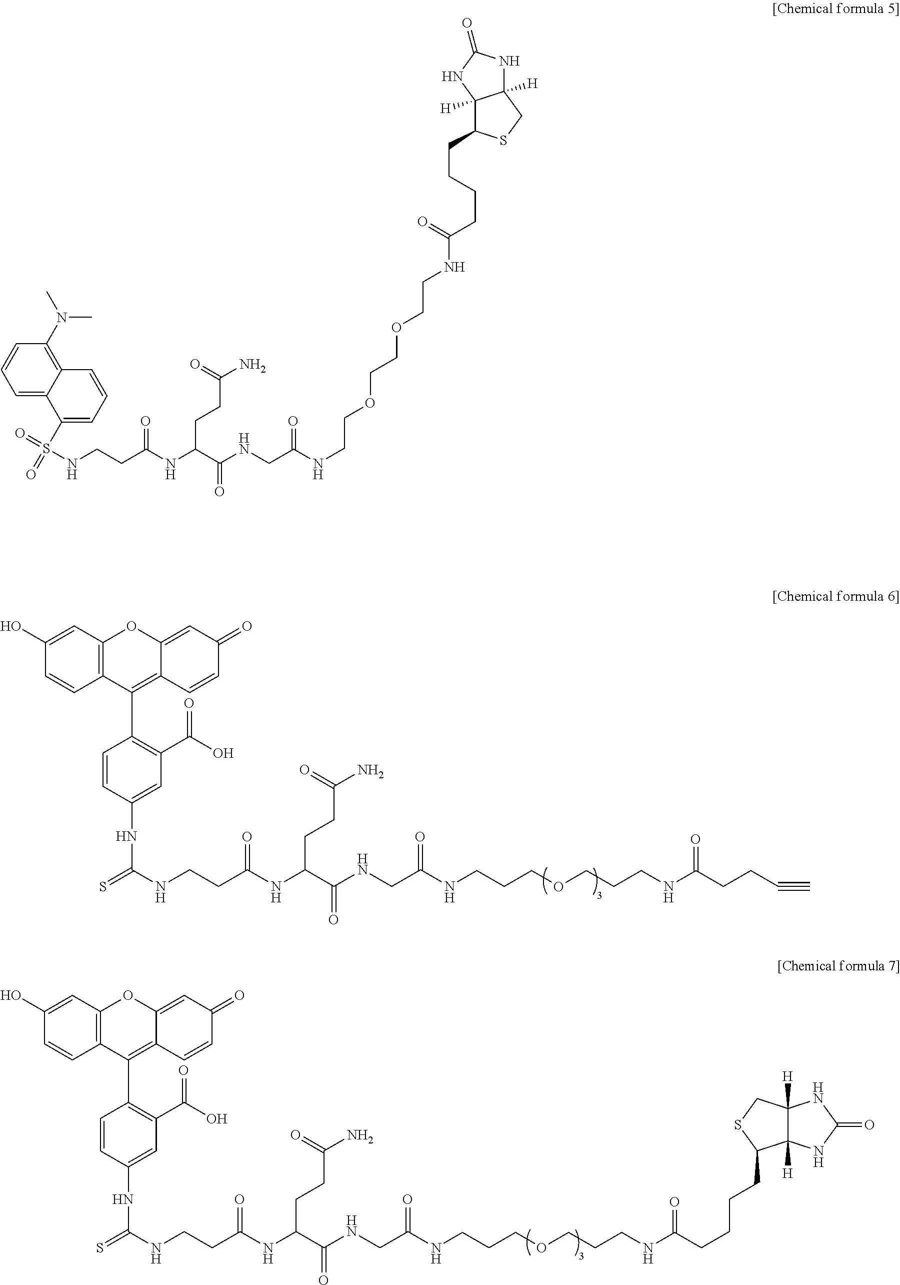

4. A fluorescently labeled protein obtained by TGase-catalyzed conjugating of a protein having a Lys residue capable of recognition by TGase and the substrate of TGase according to any one of claims 1 to 3.

5. The fluorescently labeled protein according to claim 4 wherein the protein is an IgG antibody.

6. A process for producing a fluorescently labeled protein which comprises the step of TGase-catalyzed conjugating of a protein having a Lys residue capable of recognition by TGase and the substrate of TGase according to any one of claims 1 to 3 to obtain a fluorescently labeled protein.

7. The process according to claim 6 wherein the protein is an IgG antibody.

8. A method for fluorescent labeling of a protein which comprises a step in which a functional protein having a Lys residue capable of recognition by TGase and a fluorescent group to which a portion having a Gln residue capable of recognition by TGase is bound via a linker are conjugated by TGase catalysis to obtain a fluorescently labeled functional protein with the function thereof left intact.

9. A method of labeling a protein which comprises the step of TGase-catalyzed conjugating of a protein having a Lys residue capable of recognition by TGase and a substrate of TGase represented by (fluorescent group)-(linker)-(a portion containing a Gln residue capable of recognition by TGase)-(functional organic molecule)
(wherein:
the fluorescent group is fluorescein isothiocyanate (FITC), Texas Red (1E) or dansyl (Dns) or a group derived therefrom;
the linker is a group represented by —NH—(CH₂)ₙ—CO— (n is an integer of 1 to 6); and
the portion containing a Gln residue capable of recognition by TGase is QX (X is an amino acid residue other than Lys).

10. The method according to claim 9 wherein the portion containing a Gln residue capable of recognition by TGase is QG and the functional organic molecule is a group derived from biotin or alkyne.

11. The method according to claim 10 wherein the substrate of TGase is [chemical structure]
